(19) United States
(12) Patent Application Publication
(10) Pub. No.: US 2011/0206697 A1
(43) Pub. Date: Aug. 25, 2011

(54) SPINK1 TARGETED THERAPY

(75) Inventors: Arul M. Chinnaiyan, Plymouth, MI (US); Bushra Ateeq, Ann Arbor, MI (US); Scott Tomlins, Ann Arbor, MI (US)

(73) Assignee: The Regents of The University of Michigan, Ann Arbor, MI (US)

(21) Appl. No.: 13/023,694

(22) Filed: Feb. 9, 2011

Related U.S. Application Data
(60) Provisional application No. 61/306,267, filed on Feb. 19, 2010.

(51) Int. Cl. A61K 39/395; C12N 5/071; C12N 5/09; A61P 35/00; C07K 16/18

(52) U.S. Cl. 424/172.1; 435/375; 530/389.8

(57) ABSTRACT

The present invention relates to compositions and methods for cancer therapy, including but not limited to, targeted inhibition of cancer markers. In particular, the present invention relates to SPINK1 as a clinical target for prostate cancer.

This application claims priority to provisional application 61/306,267, filed Feb. 19, 2010, which is herein incorporated by reference in its entirety.

### STATEMENT REGARDING FEDERALLY SPONSORED RESEARCH OR DEVELOPMENT

This invention was made with government support under Grant numbers CA069568, CA132874 and CA111275 awarded by the National Institutes of Health and W81XWH-08-1-003 awarded by the Army Medical Research and Material Command. The government has certain rights in the invention.

### FIELD OF THE INVENTION

The present invention relates to compositions and methods for cancer therapy, including but not limited to, targeted inhibition of cancer markers. In particular, the present invention relates to SPINK1 as a clinical target for prostate cancer.

### BACKGROUND OF THE INVENTION

Prostate cancer is the most common nondermatologic cancer and the second most common cause of cancer-related deaths in American men. The number of prostate cancers recorded in cancer registries in the United States and the United Kingdom has increased markedly in the past 15 years. This change predominantly represents an increase in the number of cancers diagnosed rather than a real increase in the number of cancers in the population. In 2006, 234,460 new cases and 27,350 deaths were estimated to occur. It was determined that approximately 91% of these new cases would be diagnosed at local or regional stages.

Prostate cancer (PCa) is typically diagnosed with a digital rectal exam and/or prostate specific antigen (PSA) screening. An elevated serum PSA level can indicate the presence of PCa. PSA is used as a marker for prostate cancer because it is secreted only by prostate cells. A healthy prostate will produce a stable amount—typically below 4 nanograms per milliliter, or a PSA reading of “4” or less—whereas cancer cells produce escalating amounts that correspond with the severity of the cancer. A level between 4 and 10 may raise a doctor's suspicion that a patient has prostate cancer, while amounts above 50 may show that the tumor has spread elsewhere in the body.

When PSA or digital tests indicate a strong likelihood that cancer is present, a transrectal ultrasound (TRUS) is used to map the prostate and show any suspicious areas. Biopsies of various sectors of the prostate are used to determine if prostate cancer is present. Treatment options depend on the stage of the cancer. Men with a 10-year life expectancy or less who have a low Gleason number and whose tumor has not spread beyond the prostate are often treated with watchful waiting (no treatment). Treatment options for more aggressive cancers include surgical treatments, such as radical prostatectomy (RP) in which the prostate is completely removed (with or without nerve sparing techniques), and radiation, applied through an external beam that directs the dose to the prostate from outside the body or via low-dose radioactive seeds that are implanted within the prostate to kill cancer cells locally. Anti-androgen hormone therapy is also used, alone or in conjunction with surgery or radiation. Hormone therapy uses luteinizing hormone-releasing hormones (LH-RH) analogs, which block the pituitary from producing hormones that stimulate testosterone production. Patients must have injections of LH-RH analogs for the rest of their lives.

While surgical and hormonal treatments are often effective for localized PCa, advanced disease remains essentially incurable. Androgen ablation is the most common therapy for advanced PCa, leading to massive apoptosis of androgen-dependent malignant cells and temporary tumor regression. In most cases, however, the tumor reemerges with a vengeance and can proliferate independent of androgen signals.

Thus, additional therapies that target prostate cancers are needed.

### SUMMARY OF THE INVENTION

The present invention relates to compositions and methods for cancer therapy, including but not limited to, targeted inhibition of cancer markers. In particular, the present invention relates to SPINK1 as a clinical target for prostate cancer.

For example, in some embodiments, the present invention provides a method of inhibiting at least one biological function of SPINK1 (e.g., SPINK secreted by a cell) comprising contacting the SPINK1 with a reagent that inhabits at least one biological function of the SPINK1. In some embodiments, the method comprises contacting a SPINK1 polypeptide with an antibody that specifically binds to the SPINK1 polypeptide and inhibits at least one biological function of the SPINK1 polypeptide (e.g., those described herein). In some embodiments, the cell is a cancer cell (e.g., a prostate cancer cell). In some embodiments, the method comprises administering a nucleic acid based therapeutic (e.g., siRNA, antisense and the like) that inhibits expression of a SPINK1 mRNA or polypeptide. In some embodiments, the cell is in vivo, in vitro, ex vivo, or in an animal (e.g., a human or a non-human mammal). In some embodiments, the cell does not harbor an ETS gene fusion (e.g., a TMPRSS2:ETS fusion). In some embodiments, the reagent inhibits the proliferation or the invasiveness of the cell. In some embodiments, one or more chemotherapeutic agents are administered in combination with the antibody.

Further embodiments provide a method of inhibiting at least one biological function of EGFR, for example, alone or in combination with inhibition of a biological function of SPINK. In some embodiments, an siRNA reagent, antisense reagent or a monoclonal antibody is used to inhibit a biological function of EGFR.

In additional embodiments, the present invention provides a kit, comprising a pharmaceutical composition that inhibits at least one biological function of SPINK1 and/or EGFR (e.g., SPINK secreted by a cell, wherein the cell is located in vivo, ex vivo, in vitro or in an animal). In some embodiments, the composition comprises a nucleic acid based therapeutic (e.g., siRNA, antisense or the like) or an antibody (e.g. an antibody that specifically binds to SPINK1 and inhibits at least one biological function of SPINK1).

Additional embodiments are described herein.

### DESCRIPTION OF THE FIGURES

FIG. 1 shows that SPINK1 promotes cell proliferation and invasion in SPINK1 negative cell lines. (A) SPINK1 stimulates cell proliferation in SPINK1⁻/ETS⁻ cell lines. (B) SPINK1 mediates invasion of benign immortalized prostate cell line RWPE as measured by Boyden chamber Matrigel invasion assay. (C) 22RV1 cells transfected with siRNA against SPINK1 showed decrease in cell invasion.

FIG. 2 shows that stable knockdown of SPINK1 inhibits cell proliferation and invasion of SPINK1⁺ prostate cancer cells. (A) SPINK1 knockdown in 22RV1 cells was confirmed at the transcript level by quantitative PCR and protein level by immunofluorescence (upper insets; 600× magnification) using an antibody against SPINK1. (B) Boyden chamber Matrigel invasion assay demonstrates decrease in cell invasion in stable pooled shSPINK1 knockdown 22RV1 cells as compared to pooled shNS vector cells. (C) Cell proliferation assay showing decrease in proliferation in pooled or single clone shSPINK1 knockdown 22RV1 cells as compared to pooled shNS vector cells at the indicated time points. (D) Soft agar colony assay showed decrease in the number of colonies in stable pooled shSPINK1 knockdown 22RV1 cells as compared to pooled shNS vector cells.

FIG. 3 shows that Anti-SPINK1 mAb attenuates in vitro proliferation and invasion exclusively in SPINK1+/ETS-prostate cancer cells. (A) Cell proliferation of DU145, PC3 and 22RV 1 cells was assessed in the presence of 1 μg/ml SPINK1 mAb or IgG mAb (B) As in A except using 22RV1 cells and 0.5-1 μg/ml SPINK1 mAb or IgG mAb. (C) Effect of SPINK1 mAb or IgG mAb on invasion of SPINK1+/ETS-cells (22RV 1 and CWR22PC) and SPINK1+/ETS-cells (DU145, PC3, LNCaP and VCaP).

FIG. 4 shows SPINK1 as a therapeutic target in SPINK1⁺ prostate cancer. (A) Chick chorioallantoic membrane (CAM) assay quantifying intravasated RWPE cells upon stimulation with rSPINK1 (n=6 in each group). (B) CAM assay using 22RV1 cells in the presence of IgG mAb, SPINK1 mAb or C225 (n=5 in each group), with fold change of intravasated cells compared to IgG mAb plotted. (C) As in B, except using PC3 cells. (D) Subcutaneous xenograft growth of shNSluciferase (luc) or shSPINK1-luc 22RV1 cells implanted in male BALB/C nu/nu mice (n=10 in each group). (E) As in D, except using 22RV1-luc cell xenografts treated with control IgG mAb (n=8), SPINK1 mAb (n=6) or C225 (n=8) (10 mg/kg body weight) twice a week. (F) Same as in E, except mice (n=7 per group) were treated with a combination of SPINK1 mAb and C225 mAb (10 mg/kg body weight for both). (G) As in E & F, except using PC3-luc xenografts treated with control IgG mAb, SPINK1 mAb or C225 (n=8 per group) (10 mg/kg body weight) alone or in combination twice a week. (H) Representative bioluminescence images from mice in D bearing pooled shNS-luc or shSPINK1-luc xenografts and % reduction in tumor volume at week 5. (I) Same as H, except bioluminescence images from mice bearing 22RV1-luc xenografts in (top panel) or PC3-luc (lower panel) mice treated with IgG mAb, SPINK1 mAb, or C225 mAb alone or in combination, with comparative % reduction plot in tumor volume at week 5.

FIG. 5 shows bacterial expression vectors (pQE-9) constructed to produce N-6× His-tag SPINK1 recombinant protein (rSPINK1) from human cDNA.

FIG. 6 shows the effect of conditioned medium (CM) collected from 22RV1 cells (10 kd fraction) or rSPINK1 protein in the presence or absence of anti-SPINK1 monoclonal antibody on breast cancer MCF7 cells invasion in Boyden chamber Matrigel invasion assay.

FIG. 7 shows the effect of conditioned medium (CM) collected from RWPE cells (10 kd fraction), multiple tag protein (including 6× His) or rSPINK1 protein on 22RV1 or RWPE cells invasion in Boyden chamber Matrigel invasion assay.

FIG. 8 shows that SPINK1 mAb reduces SPINK1+ cell motility and SPINK1 knockdown alters MAPK pathway. A, Cell motility assay was carried out by plating 22RV1 cells in the presence or absence of the SPINK1 mAb or IgG mAb on a lawn of microscopic fluorescent beads on collagen coated 96-well plates. B, Quantitative RT-PCR showing decrease in EGFR expression in the 22RV1 cells. C, Western blot showing pMEK, pERK, pAKT, tMEK, tERK and tAKT expression levels in shNS and shSPINK1 22RV1 cells (single clone). D, Same as C, except pERK and tERK levels in the 22RV1 cells treated with IgG or SPINK1 mAb. pMEK, pERK and pAKT denotes phosphorylated-MEK, -ERK or -AKT and tMEK, tERK or tAKT denotes total-MEK, -ERK or -AKT levels.

FIG. 9 shows that SPINK1 mAb induces decrease in tumor proliferation index, but has no effect on toxicity markers. A, Ki-67 immunohistochemical (IHC) staining of tumor tissue showing Ki-67 positive nuclei in SPINK1 mAb treatment group as compared to control IgG. B, Pancreatic toxicity markers showing amylase and lipase levels (U/L) in the serum samples collected from control IgG or anti-SPINK1 mAb treated mice. C, Same as B, except hepatic toxicity markers showing alkaline phosphatase (ALKP), alanine aminotranferease (ALT) and aspartate aminotransferase (AST) levels (U/L). D, Same as B, except general health profile markers showing CK: creatinine kinase (U/L); CHOL: cholesterol; TRIG: triglycerides; CREA: creatine; Ca: calcium; Mg: magnesium; PHOS: phosphorus (mg/ml).

FIG. 10 shows that Anti-IgG or -SPINK1 mAb or C225 administration has no effect on mouse body weight. A, Body weight was recorded for the mice treated with control IgG or anti-SPINK1 monoclonal antibodies. B, same as A except mice were treated with a combination of SPINK1 monoclonal antibody and C225. C, same as A except mice were xenografted with PC3 cells.

FIG. 11 shows that SPINK1 mediates its oncogenic effects in part through EGFR. (A) Immunoprecipitation using anti-IgG, anti-SPINK1 or anti-GST of exogenous SPINK1-GST, GST or GST-VEGFR added to HEK-293 cells transfected with EGFR and immunoblotted with anti-EGFR (top panel), and immunoprecipitation using anti-IgG or anti-SPINK1 of exogenous SPINK1-GST added to 22RV1 cells and immunoblotted with anti-EGFR (bottom panel). (B) Western blot showing EGFR phosphorylation in response to rSPINK1 (100 ng/ml) or EGF (10 ng/ml) stimulation. (C) Invasion assay showing siRNA mediated EGFR knockdown 22RV1 cells treated with 10 ng/ml of rSPINK1 (D) Same as in C, except with RWPE cells. (E) Invasion assay showing rSPINK1 (10 ng/ml) stimulated RWPE cells in the presence or absence of C225 (cetuximab, 50 μg/ml) or IgG mAb (50 μg/ml) (F) Invasion assay showing the effect of IgG or C225 antibody on SPINK1+ and SPINK1− cancer cells. (G) As in F, except 22RV1 cells were treated with a combination of anti-SPINK1 and/or C225 mAb (1 μg/ml and 50 μg/ml respectively). (H) Cell proliferation assay using the indicated cells in the presence of IgG mAb or C225.

FIG. 12 shows expression of SPINK1 in a prostate cell line panel by quantitative RT-PCR.

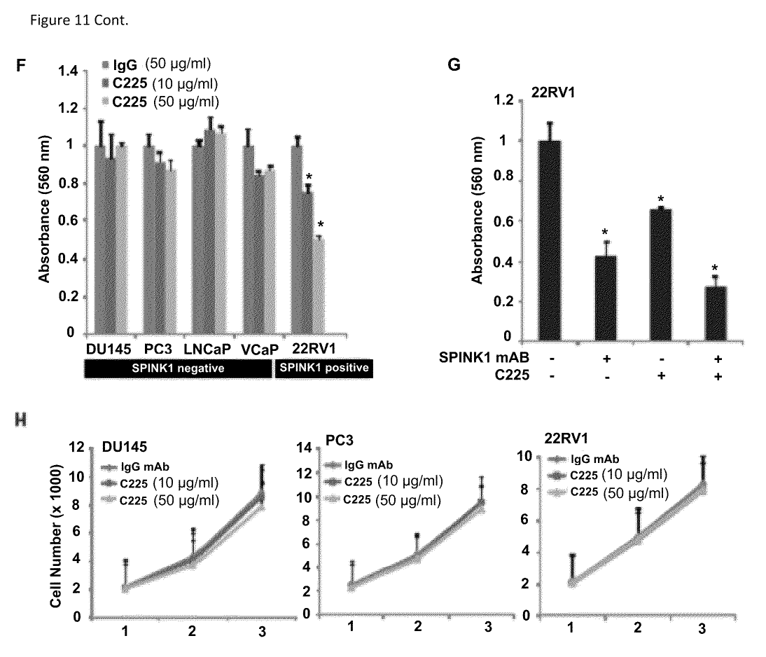

FIG. 13 shows that CM collected from 22RV1 cells induces cell invasion, but not CM from LNCaP cells.

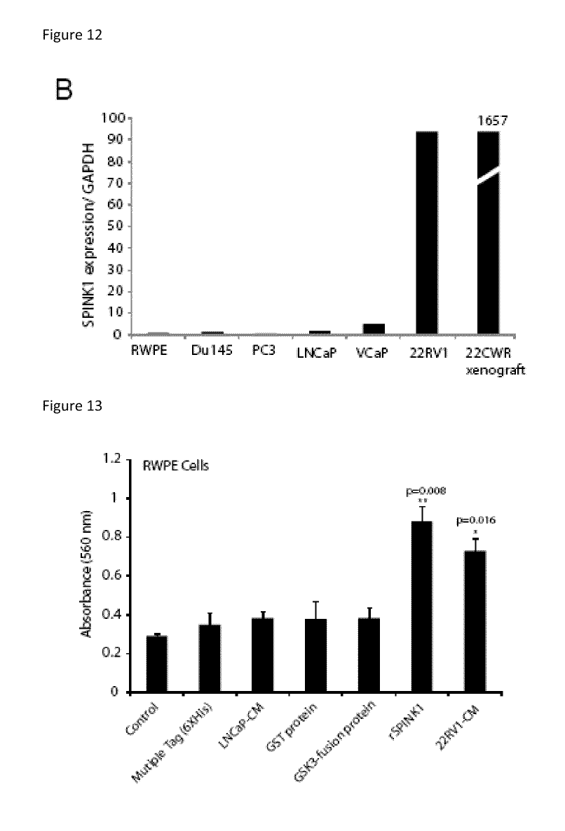

FIG. 14 shows that PRSS1 (trypsin1) knockdown in 22RV1 cells has no effect on SPINK1 mediated cell invasion. A, Expression of PRSS1 (trypsin1) by qPCR. Pancreatic cancer cells, CAPAN1 was used as a control. B, Same as A except SPINK1 was knocked down in the 22RV1 cell line using siRNA against SPINK1. C, Western blot showing trypsin levels in the 22RV1 cells stimulated with rSPINK1 or EGF at different time points as indicated. D-E, PRSS1 was knocked down in the 22RV1 cell line using multiple siRNA constructs. D, PRSS1 expression was determined by qPCR and E, the effect on invasion was determined by Boyden chamber Matrigel invasion assay.

FIG. 15 shows that exogenous rSPINK1 has no effect on PSA in 22RV1 cells. A, Western blot showing no change in PSA level in 22RV1 cell line stimulated with rSPINK1 (100 ng/ml) or EGF (10 ng/ml). B, Matrigel invasion assay using 22RV1 cell line in the presence of IgG or PSA monoclonal antibody.

FIG. 16 show that exogenous SPINK1 induces EGFR dimerization and phosphorylation. A, Western blot showing EGFR phosphorylation in the stable shNS, shSPINK1 pool and in single shSPINK1 clone. B, Non-reducing Western blot showing EGFR dimerization after stimulating with rSPINK1 (100 ng/ml) and EGF (10 ng/ml) as indicated in the presence or absence of crosslinking reagent BS3 (3 mM in PBS).

FIG. 17 shows mapping of the epitope on the SPINK1 monoclonal antibody.

### DEFINITIONS

To facilitate an understanding of the present invention, a number of terms and phrases are defined below:

As used herein, the term “inhibits at least one biological activity of serine peptidase inhibitor, Kasal type I (SPINK1)” refers to any agent that decreases any activity of SPINK1 (e.g., including, but not limited to, the activities described herein), via directly contacting SPINK1 protein, contacting SPINK1 mRNA or genomic DNA, causing conformational changes of SPINK1 polypeptides, decreasing SPINK1 protein levels, or interfering with SPINK1 interactions with signaling partners, and affecting the expression of SPINK1 target genes. Inhibitors also include molecules that indirectly regulate SPINK1 biological activity by intercepting upstream signaling molecules.

As used herein, the term “siRNAs” refers to small interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to, or substantially complementary to, a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

As used herein, the term “outlier expression of SPINK1” refers to an altered level of expression of SPINK1 nucleic acid (e.g., mRNA) or protein relative to the level normally found (e.g., the level in a subject not diagnosed with cancer). In some embodiments, normal levels are the average level in a population of one or more individuals not diagnosed with cancer. In other embodiments, normal levels are determined within other tissues of the individual to be diagnosed. In some embodiments, expression is altered by at least 10%, preferably at least 20%, even more preferably at least 50%, yet more preferably at least 75%, still more preferably at least 90%, and most preferably at least 100% relative to the level of expression normally found (e.g., in non-cancerous tissue). Expression levels may be determined using any suitable method. In some embodiments, samples positive for outlier expression of SPINK1 are those whose expression differs by greater than about 0.1, preferably greater than 0.2, and even more preferably greater than 0.5 normalized expression units. Normalized expression units may be calculated using any suitable method.

As used herein, the term “overexpression of SPINK1” refers to a higher level of expression of SPINK1 nucleic acid (e.g., mRNA) or protein relative to the level normally found. In some embodiments, expression is increased at least 10%, preferably at least 20%, even more preferably at least 50%, yet more preferably at least 75%, still more preferably at least 90%, and most preferably at least 100% relative to the level of expression normally found. The level of expression normally found may be determined using any number of suitable parameters. Examples include, but are not limited to, the level in non-cancerous prostate (e.g., an average of the level of SPINK1 expression in prostate tissues from multiple subjects not diagnosed with prostate cancer), the level in non-cancerous tissues (e.g., an average of the level of SPINK1 expression in non-prostate tissues from multiple subjects not diagnosed with cancer), the level in non-cancerous prostate cell lines, or a relative level of expression (e.g., the level over time in the same individual). Expression levels may be determined using any suitable method, including, but not limited to, those disclosed herein. In some embodiments, expression levels are compared to the level of expression of a known gene (e.g., the level of expression or the relative expression). In some embodiments, the known gene is PSA. As used herein, the term “gene expression associated with prostate cancer recurrence” refers to a gene expression profile (e.g., outlier expression of SPINK1) associated with prostate cancer recurrence (e.g., in the prostate or metastatic) following treatment (e.g., surgery) for a primary tumor. In some embodiments, prostate cancer recurrence is increased at least 10%, preferably at least 20%, even more preferably at least 50%, yet more preferably at least 75%, still more preferably at least 90%, and most preferably at least 100% relative to the level of recurrence in representative subject population (e.g., average of a large population (e.g., one or more, preferably 100 or more, even more preferably 1000 or more and still more preferably 10,000 or more subjects) of subjects lacking “outlier expression of SPINK1”).

The term “epitope” as used herein refers to that portion of an antigen that makes contact with a particular antibody.

When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “non-human animals” refers to all non-human animals including, but are not limited to, vertebrates such as rodents, non-human primates, ovines, bovines, ruminants, lagomorphs, porcines, caprines, equines, canines, felines, ayes, etc.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using the screening methods described herein. In some embodiments, test compounds include antisense compounds.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the described compositions and methods.

As used herein, the term “prostate sample” refers to any sample containing prostate cells or secretions. Example of prostate samples include, but are not limited to, a prostate tissue sample (e.g., a biopsy sample) or a urine sample.

### DETAILED DESCRIPTION OF THE INVENTION

The present invention relates to compositions and methods for cancer therapy, including but not limited to, targeted inhibition of cancer markers. In particular, the present invention relates to SPINK1 as a clinical target for prostate cancer.

When applied to the Oncomine database (Rhodes et al., Proc Natl Acad Sci USA 101, 9309 [2004]; Rhodes et al., Neoplasia 6, 1 [2004]), the methodology termed Cancer Outlier Profile Analysis (COPA) correctly identified several known oncogenes, including PBX1 in leukemia and CCND1 in multiple myeloma (Tomlins et al., Science 310, 644 [2005]). In addition, COPA nominated ETS family genes as candidate oncogenes in prostate cancer prompting the discovery of recurrent chromosomal rearrangements involving ERG or ETV1 and the androgen-regulated gene TMPRSS2 (Tomlins et al., [2005], supra).

As 50-70% of prostate cancers harbor TMPRSS2:ETS fusions, experiments were conducted to identify additional candidate oncogenes in prostate cancers. Experiments conducted used a meta-analysis of COPA applied to 7 prostate cancer profiling studies and analyzed candidates for outlier expression in prostate cancer and mutually exclusive over-expression with ERG and ETV1. SPINK1, which was identified as the 2nd ranked meta-outlier, met both criteria across 8 data sets. SPINK1 showed marked overexpression in 50 of 325 (15.4%) profiled prostate cancers, but only 1 of 56 (1.8%) benign prostate tissue samples. In all 325 profiled prostate cancer samples, SPINK1, ERG and ETV 1 showed mutually exclusive outlier expression. The over-expression of SPINK1 in a fraction of cancer samples compared to benign prostate tissue and the mutually exclusive over-expression of SPINK1, ERG and ETV1 was confirmed by quantitative PCR. Fluorescence in situ hybridization from tissue from one of the localized prostate cancers over-expressing SPINK1 did not reveal gene rearrangements or amplification, indicating that SPINK1 is up-regulated through increased transcription. Together these results, consistent across different assays, microarray platforms, laboratories and sample cohorts, demonstrate that SPINK1 is exclusively over-expressed in prostate cancers without TMPRSS2:ETS SPINK1s (as indicated by ERG or ETV1 over-expression).

The cDNA sequence of SPINK1 (serine peptidase inhibitor, Kazal type 1) is provided in Genbank accession number NM_{—}003122.2. The peptide encoded by SPINK1, also known as PSTI or TATI, was originally isolated from bovine pancreas and human pancreatic juice; its normal function is thought to be the inhibition of trypsin in the pancreas (Haverback et al., Am J Med 29, 421-33 (1960); Kazal et al., Journal of the American Chemical Society 70, 3034-3040 (1948); Paju et al., Crit Rev Clin Lab Sci 43, 103-42 (2006); Greene et al., Methods Enzymol 45, 813-25 (1976)). SPINK1 mRNA and protein have been detected in a variety of benign and cancerous tissues, however its expression in prostate has not been described (reviewed in Paju and Stenman, Crit Rev Clin Lab Sci 43, 103-42 (2006), Stenman, Clin Chem 48, 1206-9 (2002)). SPINK1 encodes a 79 amino acid peptide with a 23 amino acid signal peptide and is detectable in the urine and serum of healthy individuals (Paju and Stenman, supra). In addition to being strongly elevated during severe inflammation and pancreatitis, serum levels of SPINK1 may be dysregulated in numerous cancers, including pancreatic, gastric, liver, lung, breast, bladder, renal, head and neck, colorectal, kidney and ovarian cancer (reviewed in Paju and Stenman, supra, Stenman, supra).

The mouse homologue (SPINK3), plays important roles in proliferation and differentiation of various cell types during embryonic development (Wang et al., Histochem Cell Biol 130, 387-397 (2008)). Apart from its normal expression in pancreatic acinar cells, SPINK1 mRNA or protein is often expressed in various types of human cancers (Kelloniemi et al., Urology 62, 249-253 (2003); Lukkonen et al., Int J Cancer 83, 486-490 (1999); Haglund et al., Br J Cancer 54, 297-303 (1986); Higashiyama et al., Br J Cancer 62, 954-958 (1990); Huhtala et al., Int J Cancer 31, 711-714 (1983); Paju et al., Clin Cancer Res 10, 4761-4768 (2004); Ohmachi et al., Int J Cancer 55, 728-734 (1993)), and increased serum SPINK1 level correlates with poor prognosis in some studies (Kelloniemi et al., supra; Lukkonen et al., supra; Paju et al., supra). The prostate gland also secretes a variety of serine proteases, most notably the kallikrein enzyme PSA, but also trypsin, which is over-expressed in prostate cancer (Bjartell et al., Prostate 64, 29-39 (2005)).

Therapies targeted against specific molecular alterations present only in cancer cells have revolutionized treatment in several cancers, such as imatinib (Gleevec), which targets the BCR-ABL chimeric protein in chronic myelogenous leukemia and trastuzumab (Herceptin) targeting ERBB2, which is amplified in ˜25% of breast cancers. In prostate cancer, although multiple currently approved therapies (and newer agents in late stage development) target the androgen signaling axis, additional targeted therapies are lacking SPINK1 encodes a cell surface anti-proteinase, which may be amendable to therapeutic targeting by traditional strategies, such as monoclonal antibodies.

Experiments conducted during the course of development of embodiments of the present invention demonstrated that SPINK1 promotes prostate cancer proliferation and invasion through autocrine and paracrine signaling. Mutation of SPINK1 at leucine 18 (L18) in the trypsin interaction site reduced tumor growth, angiogenesis and lung metastases in HT-29 5M21 human colon carcinoma tumor xenografts, indicating that the cancer related phenotypes of SPINK1 may be related to its anti-proteinase activity (Gouger et al., Oncogene 27, 4024-4033 (2008)). Additionally, SPINK1 has been shown to engage the EGFR/mitogen-activated protein kinase cascade in NIH3T3 fibroblasts and pancreatic cancer cells (Ozaki et al., Mol Cancer Res 7, 1572-1581 (2009)). SPINK1 was also discovered as an apoptosis inhibitor preventing the serine protease dependent cell apoptosis of malignant cells (Lu et al., Apoptosis 13, 483-494 (2008)). The present invention is not limited to a particular mechanism. Indeed, an understanding of the mechanism is not necessary to practice the present invention. Nonetheless, it is contemplated that this finding in corroboration with the experiments described herein indicates that SPINK1 not only acts as pro-proliferation and pro-invasive autocrine, but can also make cancer cells resistant to apoptosis and acts as an important factor in cancer progression.

Discovery of hormone-driven expression of the ERG after fusion with TMPRSS2 (Tomlins et al., Science 310, 644-648 (2005)), allows for the treatment of androgen driven ETS-positive patients with anti-androgen agents such as abiraterone acetate or CYP17 specific inhibitor, which is known to ablate the synthesis of androgens and estrogens that drive TMPRSS2-ERG fusions (Setlur et al., J Natl Cancer Inst 100, 815-825 (2008)). Other less common hormone-dependent fusions of ETV1, ETV4, and ETV5 could also account for the abiraterone-sensitive cancers (Helgeson et al., Cancer Res 68, 73-80 (2008); Tomlins et al., Science 310, 644-648 (2005); Tomlins et al., Cancer Res 66, 3396-3400 (2006)). Beside novel anti-androgens that target the AR, small molecule inhibitors of the phosphatidylinositol-3-kinase (PI3K) are also being evaluated in clinical trials as a strategy for reversing resistance to hormone therapies (Attard et al., Br J Cancer 95, 767-774 (2006)). Nevertheless, all newly emerging anti-androgen therapies are targeted against ETS-rearrangement positive subset only (30-70% of cases), leaving 10-15% of SPINK1⁻ ETS-negative prostate cancer cases without any effective therapy.

Experiments conducted during the course of development of embodiments of the present invention demonstrated that targeting SPINK1, either by stable shRNA or an anti-SPINK1 mAb, resulted in decreased tumor growth in 22RV1 (SPINK1⁺/ETS⁻) xenograft models. These results demonstrate both the importance of identifying molecular subtypes in prostate cancer and testing potential therapies in appropriate model systems representing such molecular subtypes. Indeed, targeting SPINK1 in LNCaP (SPINK1⁻/ETS⁺) or PC3 (SPINK1⁻/ETS⁻) cells, the most commonly used lines for preclinical testing of therapies for prostate cancer, had no effect in in vitro studies described here and previously (Tomlins et al., Cancer Cell 13, 519-528 (2008)).

### I. Therapeutic Applications

In some embodiments, the present invention provides therapies for cancer (e.g., prostate cancer). In some embodiments, therapies directly or indirectly target SPINK1. In some embodiments, the therapies target SPINK1 cancer cells that do not harbor an ETS gene fusion (e.g., a TMPRSS2:ETS gene fusion).

In some embodiments, therapies target epidermal growth factor receptor (EGFR), either directly or indirectly. Experiments conducted during the course of development of embodiments of the present invention demonstrated that co-targeting of both SPINK1 and EGFR resulted in enhanced and additive attenuation of tumor growth. Thus, in some embodiments, a combination of a therapeutic that targets SPINK1 and a second therapeutic that targets EGFR is utilized.

A. Antibody Therapy

In some embodiments, the present invention provides antibodies that target SPINK1 and/or EGFR. Any suitable antibody (e.g., monoclonal, polyclonal, or synthetic) may be utilized in the therapeutic methods disclosed herein. In some embodiments, the antibodies described in the experimental section below are utilized. In some embodiments, commercially available antibodies are utilized (e.g., from Mobitec, Göttingen, Germany). In preferred embodiments, the antibodies used for cancer therapy are humanized antibodies. Methods for humanizing antibodies are well known in the art (See e.g., U.S. Pat. Nos. 6,180,370, 5,585,089, 6,054,297, and 5,565,332; each of which is herein incorporated by reference).

In some embodiments, the therapeutic antibodies comprise an antibody generated against SPINK1 and/or EGFR, wherein the antibody is conjugated to a cytotoxic agent. In such embodiments, a tumor specific therapeutic agent is generated that does not target normal cells, thus reducing many of the detrimental side effects of traditional chemotherapy. For certain applications, it is envisioned that the therapeutic agents will be pharmacologic agents that will serve as useful agents for attachment to antibodies, particularly cytotoxic or otherwise anticellular agents having the ability to kill or suppress the growth or cell division of endothelial cells. The present invention contemplates the use of any pharmacologic agent that can be conjugated to an antibody, and delivered in active form. Exemplary anticellular agents include chemotherapeutic agents, radioisotopes, and cytotoxins. The therapeutic antibodies of the present invention may include a variety of cytotoxic moieties, including but not limited to, radioactive isotopes (e.g., iodine-131, iodine-123, technicium-99m, indium-111, rhenium-188, rhenium-186, gallium-67, copper-67, yttrium-90, iodine-125 or astatine-211), hormones such as a steroid, antimetabolites such as cytosines (e.g., arabinoside, fluorouracil, methotrexate or aminopterin; an anthracycline; mitomycin C), vinca alkaloids (e.g., demecolcine; etoposide; mithramycin), and antitumor alkylating agent such as chlorambucil or melphalan. Other embodiments include agents such as a coagulant, a cytokine, growth factor, bacterial endotoxin or the lipid A moiety of bacterial endotoxin. For example, in some embodiments, therapeutic agents include plant-, fungus- or bacteria-derived toxin, such as an A chain toxins, a ribosome inactivating protein, α-sarcin, aspergillin, restrictocin, a ribonuclease, diphtheria toxin or pseudomonas exotoxin, to mention just a few examples. In some preferred embodiments, deglycosylated ricin A chain is utilized.

In any event, it is proposed that agents such as these may, if desired, be successfully conjugated to an antibody, in a manner that will allow their targeting, internalization, release or presentation to blood components at the site of the targeted tumor cells as required using known conjugation technology (See, e.g., Ghose et al., Methods Enzymol., 93:280 [1983]).

For example, in some embodiments the present invention provides immunotoxins targeting SPINK1 and/or EGFR. Immunotoxins are conjugates of a specific targeting agent typically a tumor-directed antibody or fragment, with a cytotoxic agent, such as a toxin moiety. The targeting agent directs the toxin to, and thereby selectively kills, cells carrying the targeted antigen. In some embodiments, therapeutic antibodies employ crosslinkers that provide high in vivo stability (Thorpe et al., Cancer Res., 48:6396 [1988]).

In other embodiments, particularly those involving treatment of solid tumors, antibodies are designed to have a cytotoxic or otherwise anticellular effect against the tumor vasculature, by suppressing the growth or cell division of the vascular endothelial cells. This attack is intended to lead to a tumor-localized vascular collapse, depriving the tumor cells, particularly those tumor cells distal of the vasculature, of oxygen and nutrients, ultimately leading to cell death and tumor necrosis.

In preferred embodiments, antibody based therapeutics are formulated as pharmaceutical compositions as described below. In preferred embodiments, administration of an antibody composition of the present invention results in a measurable decrease in cancer (e.g., decrease or elimination of tumor).

B. RNA Interference and Antisense Therapies

In some embodiments, the present invention targets the expression of SPINK1 and/or EGFR. For example, in some embodiments, the present invention employs compositions comprising oligomeric antisense or RNAi compounds, particularly oligonucleotides (e.g., those described herein), for use in modulating the function of nucleic acid molecules encoding SPINK1 and/or EGFR, ultimately modulating the amount of SPINK1 and/or EGFR expressed.

1. RNA Interference (RNAi)

In some embodiments, RNAi is utilized to inhibit SPINK1 and/or EGFR protein function. RNAi represents an evolutionary conserved cellular defense for controlling the expression of foreign genes in most eukaryotes, including humans. RNAi is typically triggered by double-stranded RNA (dsRNA) and causes sequence-specific mRNA degradation of single-stranded target RNAs homologous in response to dsRNA. The mediators of mRNA degradation are small interfering RNA duplexes (siRNAs), which are normally produced from long dsRNA by enzymatic cleavage in the cell. siRNAs are generally approximately twenty-one nucleotides in length (e.g. 21-23 nucleotides in length), and have a base-paired structure characterized by two nucleotide 3′-overhangs. Following the introduction of a small RNA, or RNAi, into the cell, it is believed the sequence is delivered to an enzyme complex called RISC (RNA-induced silencing complex). RISC recognizes the target and cleaves it with an endonuclease. It is noted that if larger RNA sequences are delivered to a cell, RNase III enzyme (Dicer) converts longer dsRNA into 21-23 nt ds siRNA fragments.

Chemically synthesized siRNAs have become powerful reagents for genome-wide analysis of mammalian gene function in cultured somatic cells. Beyond their value for validation of gene function, siRNAs also hold great potential as gene-specific therapeutic agents (Tuschl and Borkhardt, Molecular Intervent. 2002; 2(3):158-67, herein incorporated by reference).

The transfection of siRNAs into animal cells results in the potent, long-lasting post-transcriptional silencing of specific genes (Caplen et al, Proc Natl Acad Sci U.S.A. 2001; 98: 9742-7; Elbashir et al., Nature. 2001; 411:494-8; Elbashir et al., Genes Dev. 2001;15: 188-200; and Elbashir et al., EMBO J. 2001; 20: 6877-88, all of which are herein incorporated by reference). Methods and compositions for performing RNAi with siRNAs are described, for example, in U.S. Pat. No. 6,506,559, herein incorporated by reference.

siRNAs are extraordinarily effective at lowering the amounts of targeted RNA, and by extension proteins, frequently to undetectable levels. The silencing effect can last several months, and is extraordinarily specific, because one nucleotide mismatch between the target RNA and the central region of the siRNA is frequently sufficient to prevent silencing (Brummelkamp et al, Science 2002; 296:550-3; and Holen et al, Nucleic Acids Res. 2002; 30:1757-66, both of which are herein incorporated by reference).

An important factor in the design of siRNAs is the presence of accessible sites for siRNA binding. Bahoia et al., (J. Biol. Chem., 2003; 278: 15991-15997; herein incorporated by reference) describe the use of a type of DNA array called a scanning array to find accessible sites in mRNAs for designing effective siRNAs. These arrays comprise oligonucleotides ranging in size from monomers to a certain maximum, usually Comers, synthesized using a physical barrier (mask) by stepwise addition of each base in the sequence. Thus the arrays represent a full oligonucleotide complement of a region of the target gene. Hybridization of the target mRNA to these arrays provides an exhaustive accessibility profile of this region of the target mRNA. Such data are useful in the design of antisense oligonucleotides (ranging from 7 mers to 25 mers), where it is important to achieve a compromise between oligonucleotide length and binding affinity, to retain efficacy and target specificity (Sohail et al, Nucleic Acids Res., 2001; 29(10): 2041-2045). Additional methods and concerns for selecting siRNAs are described for example, in WO 05054270, WO05038054A1, WO03070966A2, J Mol Biol. 2005 May 13; 348(4):883-93, J Mol Biol. 2005 May 13; 348(4):871-81, and Nucleic Acids Res. 2003 Aug. 1; 31(15):4417-24, each of which is herein incorporated by reference in its entirety. In addition, software (e.g., the MWG online siMAX siRNA design tool) is commercially or publicly available for use in the selection of siRNAs.

In some embodiments, the present invention utilizes siRNA including blunt ends (See e.g., US20080200420, herein incorporated by reference in its entirety), overhangs (See e.g., US20080269147A1, herein incorporated by reference in its entirety), locked nucleic acids (See e.g., WO2008/006369, WO2008/043753, and WO2008/051306, each of which is herein incorporated by reference in its entirety). In some embodiments, siRNAs are delivered via gene expression or using bacteria (See e.g., Xiang et al., Nature 24: 6 (2006) and W006066048, each of which is herein incorporated by reference in its entirety).

In other embodiments, shRNA techniques (See e.g., 20080025958, herein incorporated by reference in its entirety) are utilized. A small hairpin RNA or short hairpin RNA (shRNA) is a sequence of RNA that makes a tight hairpin turn that can be used to silence gene expression via RNA interference. shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs which match the siRNA that is bound to it. shRNA is transcribed by RNA polymerase III.

2. Antisense

In other embodiments, SPINK1 and/or EGFR expression is modulated using antisense compounds that specifically hybridize with one or more nucleic acids encoding SPINK1 and/or EGFR. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA. The overall effect of such interference with target nucleic acid function is modulation of the expression of cancer markers of the present invention. In the context of the present invention, “modulation” means either an increase (stimulation) or a decrease (inhibition) in the expression of a gene. For example, expression may be inhibited to prevent tumor proliferation.

It is preferred to target specific nucleic acids for antisense. “Targeting” an antisense compound to a particular nucleic acid, in the context of the present invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding a SPINK1. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). Eukaryotic and prokaryotic genes may have two or more alternative start codons, any one of which may be preferentially utilized for translation initiation in a particular cell type or tissue, or under a particular set of conditions. In the context of the present invention, “start codon” and “translation initiation codon” refer to the codon or codons that are used in vivo to initiate translation of an mRNA molecule transcribed from a gene encoding a tumor antigen of the present invention, regardless of the sequence(s) of such codons.

Translation termination codon (or “stop codon”) of a gene may have one of three sequences (i.e., 5′-UAA, 5′-UAG and 5′-UGA; the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which refers to the region between the translation initiation codon and the translation termination codon, is also a region that may be targeted effectively. Other target regions include the 5′ untranslated region (5′ UTR), referring to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′ UTR), referring to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The cap region may also be a preferred target region.

Although some eukaryotic mRNA transcripts are directly translated, many contain one or more regions, known as “introns,” that are excised from a transcript before it is translated. The remaining (and therefore translated) regions are known as “exons” and are spliced together to form a continuous mRNA sequence. mRNA splice sites (i.e., intron-exon junctions) may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

In some embodiments, target sites for antisense inhibition are identified using commercially available software programs (e.g., Biognostik, Gottingen, Germany; SysArris Software, Bangalore, India; Antisense Research Group, University of Liverpool, Liverpool, England; GeneTrove, Carlsbad, Calif.). In other embodiments, target sites for antisense inhibition are identified using the accessible site method described in PCT Publ. No. WO0198537A2, herein incorporated by reference.

Once one or more target sites have been identified, oligonucleotides are chosen that are sufficiently complementary to the target (i.e., hybridize sufficiently well and with sufficient specificity) to give the desired effect. For example, in preferred embodiments of the present invention, antisense oligonucleotides are targeted to or near the start codon.

In the context of this invention, “hybridization,” with respect to antisense compositions and methods, means hydrogen bonding, which may be Watson-Crick, Hoogsteen or reversed Hoogsteen hydrogen bonding, between complementary nucleoside or nucleotide bases. For example, adenine and thymine are complementary nucleobases that pair through the formation of hydrogen bonds. It is understood that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired (i.e., under physiological conditions in the case of in vivo assays or therapeutic treatment, and in the case of in vitro assays, under conditions in which the assays are performed).

Antisense compounds are commonly used as research reagents and diagnostics. For example, antisense oligonucleotides, which are able to inhibit gene expression with specificity, can be used to elucidate the function of particular genes. Antisense compounds are also used, for example, to distinguish between functions of various members of a biological pathway.

The specificity and sensitivity of antisense is also applied for therapeutic uses. For example, antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides are useful therapeutic modalities that can be configured to be useful in treatment regimes for treatment of cells, tissues, and animals, especially humans.

While antisense oligonucleotides are a preferred form of antisense compound, the present invention comprehends other oligomeric antisense compounds. The antisense compounds in accordance with this invention preferably comprise from about 8 to about 30 nucleobases (i.e., from about 8 to about 30 linked bases), although both longer and shorter sequences may find use with the present invention. Particularly preferred antisense compounds are antisense oligonucleotides, even more preferably those comprising from about 12 to about 25 nucleobases.

Chimeric antisense compounds of the present invention may be formed as composite structures of two or more oligonucleotides, modified oligonucleotides, oligonucleosides and/or oligonucleotide mimetics as described above.

The present invention also includes pharmaceutical compositions and formulations that include the antisense compounds of the present invention as described below.

C. Genetic Therapy

The present invention contemplates the use of any genetic manipulation for use in modulating the expression of SPINK1 and/or EGFR. Examples of genetic manipulation include, but are not limited to, gene knockout (e.g., removing the SPINK1 and/or EGFR gene from the chromosome using, for example, recombination), expression of antisense constructs with or without inducible promoters, and the like. Delivery of nucleic acid construct to cells in vitro or in vivo may be conducted using any suitable method. A suitable method is one that introduces the nucleic acid construct into the cell such that the desired event occurs (e.g., expression of an antisense construct). Genetic therapy may also be used to deliver siRNA or other interfering molecules that are expressed in vivo (e.g., upon stimulation by an inducible promoter (e.g., an androgen-responsive promoter)).

Introduction of molecules carrying genetic information into cells is achieved by any of various methods including, but not limited to, directed injection of naked DNA constructs, bombardment with gold particles loaded with said constructs, and macromolecule mediated gene transfer using, for example, liposomes, biopolymers, and the like. Preferred methods use gene delivery vehicles derived from viruses, including, but not limited to, adenoviruses, retroviruses, vaccinia viruses, and adeno-associated viruses. Because of the higher efficiency as compared to retroviruses, vectors derived from adenoviruses are the preferred gene delivery vehicles for transferring nucleic acid molecules into host cells in vivo. Adenoviral vectors have been shown to provide very efficient in vivo gene transfer into a variety of solid tumors in animal models and into human solid tumor xenografts in immune-deficient mice. Examples of adenoviral vectors and methods for gene transfer are described in PCT publications WO 00/12738 and WO 00/09675 and U.S. Pat. Nos. 6,033,908, 6,019,978, 6,001,557, 5,994,132, 5,994,128, 5,994,106, 5,981,225, 5,885,808, 5,872,154, 5,830,730, and 5,824,544, each of which is herein incorporated by reference in its entirety.

Vectors may be administered to subjects in a variety of ways. For example, in some embodiments of the present invention, vectors are administered into tumors or tissue associated with tumors using direct injection. In other embodiments, administration is via the blood or lymphatic circulation (See e.g., PCT publication 99/02685 herein incorporated by reference in its entirety). Exemplary dose levels of adenoviral vector are preferably 10⁸ to 10¹¹ vector particles added to the perfusate.

D. Small Molecule Therapy

Embodiments of the present invention provide small molecules that inhibit one or more biological activities of SPINK1 and/or EGFR. Small molecule therapeutics are identified, for example, using the drug screening methods described herein.

E. Pharmaceutical Compositions

The present invention further provides pharmaceutical compositions (e.g., comprising pharmaceutical agents that modulate the expression or activity of SPINK1 and/or EGFR). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Certain embodiments of the invention provide pharmaceutical compositions containing (a) one or more antisense compounds and (b) one or more other chemotherapeutic agents that function by a non-antisense mechanism. Examples of such chemotherapeutic agents include, but are not limited to, anticancer drugs such as daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, vinblastine, etoposide, teniposide, cisplatin and diethylstilbestrol (DES). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Other non-antisense chemotherapeutic agents are also within the scope of this invention. Two or more combined compounds may be used together or sequentially.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

F. Combination Therapy

In some embodiments, the present invention provides therapeutic methods comprising one or more compositions described herein in combination with an additional agent (e.g., a chemotherapeutic agent). The present invention is not limited to a particular chemotherapy agent.

Various classes of antineoplastic (e.g., anticancer) agents are contemplated for use in certain embodiments of the present invention. Anticancer agents suitable for use with embodiments of the present invention include, but are not limited to, agents that induce apoptosis, agents that inhibit adenosine deaminase function, inhibit pyrimidine biosynthesis, inhibit purine ring biosynthesis, inhibit nucleotide interconversions, inhibit ribonucleotide reductase, inhibit thymidine monophosphate (TMP) synthesis, inhibit dihydrofolate reduction, inhibit DNA synthesis, form adducts with DNA, damage DNA, inhibit DNA repair, intercalate with DNA, deaminate asparagines, inhibit RNA synthesis, inhibit protein synthesis or stability, inhibit microtubule synthesis or function, and the like.

In some embodiments, exemplary anticancer agents suitable for use in compositions and methods of embodiments of the present invention include, but are not limited to: 1) alkaloids, including microtubule inhibitors (e.g., vincristine, vinblastine, and vindesine, etc.), microtubule stabilizers (e.g., paclitaxel (TAXOL), and docetaxel, etc.), and chromatin function inhibitors, including topoisomerase inhibitors, such as epipodophyllotoxins (e.g., etoposide (VP-16), and teniposide (VM-26), etc.), and agents that target topoisomerase I (e.g., camptothecin and isirinotecan (CPT-11), etc.); 2) covalent DNA-binding agents (alkylating agents), including nitrogen mustards (e.g., mechlorethamine, chlorambucil, cyclophosphamide, ifosphamide, and busulfan (MYLERAN), etc.), nitrosoureas (e.g., carmustine, lomustine, and semustine, etc.), and other alkylating agents (e.g., dacarbazine, hydroxymethylmelamine, thiotepa, and mitomycin, etc.); 3) noncovalent DNA-binding agents (antitumor antibiotics), including nucleic acid inhibitors (e.g., dactinomycin (actinomycin D), etc.), anthracyclines (e.g., daunorubicin (daunomycin, and cerubidine), doxorubicin (adriamycin), and idarubicin (idamycin), etc.), anthracenediones (e.g., anthracycline analogues, such as mitoxantrone, etc.), bleomycins (BLENOXANE), etc., and plicamycin (mithramycin), etc.; 4) antimetabolites, including antifolates (e.g., methotrexate, FOLEX, and MEXATE, etc.), purine antimetabolites (e.g., 6-mercaptopurine (6-MP, PURINETHOL), 6-thioguanine (6-TG), azathioprine, acyclovir, ganciclovir, chlorodeoxyadenosine, 2-chlorodeoxyadenosine (CdA), and 2′-deoxycoformycin (pentostatin), etc.), pyrimidine antagonists (e.g., fluoropyrimidines (e.g., 5-fluorouracil (ADRUCIL), 5-fluorodeoxyuridine (FdUrd) (floxuridine)) etc.), and cytosine arabinosides (e.g., CYTOSAR (ara-C) and fludarabine, etc.); 5) enzymes, including L-asparaginase, and hydroxyurea, etc.; 6) hormones, including glucocorticoids, antiestrogens (e.g., tamoxifen, etc.), nonsteroidal antiandrogens (e.g., flutamide, etc.), and aromatase inhibitors (e.g., anastrozole (ARIMIDEX), etc.); 7) platinum compounds (e.g., cisplatin and carboplatin, etc.); 8) monoclonal antibodies conjugated with anticancer drugs, toxins, and/or radionuclides, etc.; 9) biological response modifiers (e.g., interferons (e.g., IFN-α, etc.) and interleukins (e.g., IL-2, etc.), etc.); 10) adoptive immunotherapy; 11) hematopoietic growth factors; 12) agents that induce tumor cell differentiation (e.g., all-trans-retinoic acid, etc.); 13) gene therapy techniques; 14) antisense therapy techniques; 15) tumor vaccines; 16) therapies directed against tumor metastases (e.g., batimastat, etc.); 17) angiogenesis inhibitors; 18) proteosome inhibitors (e.g., VELCADE); 19) inhibitors of acetylation and/or methylation (e.g., HDAC inhibitors); 20) modulators of NF kappa B; 21) inhibitors of cell cycle regulation (e.g., CDK inhibitors); 22) modulators of p53 protein function; and 23) radiation.

Any oncolytic agent that is routinely used in a cancer therapy context finds use in the compositions and methods of embodiments of the present invention. For example, the U.S. Food and Drug Administration maintains a formulary of oncolytic agents approved for use in the United States. International counterpart agencies to the U.S.F.D.A. maintain similar formularies. The below Table provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

| | | |
|---|---|---|
| Aldesleukin | Proleukin | Chiron Corp., Emeryville, CA |
| (des-alanyl-1, serine-125 human interleukin-2) | | |
| Alemtuzumab | Campath | Millennium and ILEX |
| (IgG1κ anti CD52 antibody) | | Partners, LP, Cambridge, MA |
| Alitretinoin | Panretin | Ligand Pharmaceuticals, Inc., |
| (9-cis-retinoic acid) | | San Diego CA |
| Allopurinol | Zyloprim | GlaxoSmithKline, Research |
| (1,5-dihydro-4 H-pyrazolo[3,4-d]pyrimidin-4-one | | Triangle Park, NC |
| monosodium salt) | | |
| Altretamine | Hexalen | US Bioscience, West |
| (N,N,N′,N′,N″,N″,-hexamethyl-1,3,5-triazine-2,4,6- | | Conshohocken, PA |
| triamine) | | |
| Amifostine | Ethyol | US Bioscience |
| (ethanethiol, 2-[(3-aminopropyl)amino]-, dihydrogen | | |
| phosphate (ester)) | | |
| Anastrozole | Arimidex | AstraZeneca Pharmaceuticals, |
| (1,3-Benzenediacetonitrile,a,a,a′,a′-tetramethyl-5-(1H- | | LP, Wilmington, DE |
| 1,2,4-triazol-1-ylmethyl)) | | |
| Arsenic trioxide | Trisenox | Cell Therapeutic, Inc., Seattle, |
| | | WA |
| Asparaginase | Elspar | Merck & Co., Inc., |
| (L-asparagine amidohydrolase, type EC-2) | | Whitehouse Station, NJ |
| BCG Live | TICE BCG | Organon Teknika, Corp., |
| (lyophilized preparation of an attenuated strain of | | Durham, NC |
| Mycobacterium bovis (Bacillus Calmette-Gukin [BCG], | | |
| substrain Montreal) | | |
| bexarotene capsules | Targretin | Ligand Pharmaceuticals |
| (4-[1-(5,6,7,8-tetrahydro-3,5,5,8,8-pentamethyl-2- | | |
| napthalenyl) ethenyl] benzoic acid) | | |
| bexarotene gel | Targretin | Ligand Pharmaceuticals |
| Bleomycin | Blenoxane | Bristol-Myers Squibb Co., |
| (cytotoxic glycopeptide antibiotics produced by | | NY, NY |
| Streptomyces verticillus; bleomycin A₂ and bleomycin | | |
| B₂) | | |
| Capecitabine | Xeloda | Roche |
| (5′-deoxy-5-fluoro-N-[(pentyloxy)carbonyl]-cytidine) | | |
| Carboplatin | Paraplatin | Bristol-Myers Squibb |
| (platinum, diammine [1,1-cyclobutanedicarboxylato(2-)- | | |
| 0,0′]-,(SP-4-2)) | | |
| Carmustine | BCNU, | Bristol-Myers Squibb |
| (1,3-bis(2-chloroethyl)-1-nitrosourea) | BiCNU | |
| Carmustine with Polifeprosan 20 Implant | Gliadel Wafer | Guilford Pharmaceuticals, |
| | | Inc., Baltimore, MD |
| Celecoxib | Celebrex | Searle Pharmaceuticals, |
| (as 4-[5-(4-methylphenyl)-3-(trifluoromethyl)-1H- | | England |
| pyrazol-1-yl] benzenesulfonamide) | | |
| Chlorambucil | Leukeran | GlaxoSmithKline |
| (4-[bis(2chlorethyl)amino]benzenebutanoic acid) | | |
| Cisplatin | Platinol | Bristol-Myers Squibb |
| (PtCl₂H₆N₂) | | |
| Cladribine | Leustatin, 2- | R. W. Johnson Pharmaceutical |
| (2-chloro-2′-deoxy-b-D-adenosine) | CdA | Research Institute, Raritan, NJ |
| Cyclophosphamide | Cytoxan, | Bristol-Myers Squibb |
| (2-[bis(2-chloroethyl)amino] tetrahydro-2H-13,2- | Neosar | |
| oxazaphosphorine 2-oxide monohydrate) | | |
| Cytarabine | Cytosar-U | Pharmacia & Upjohn |
| (1-b-D-Arabinofuranosylcytosine, C₉H₁₃N₃O₅) | | Company |
| cytarabine liposomal | DepoCyt | Skye Pharmaceuticals, Inc., |
| | | San Diego, CA |
| Dacarbazine | DTIC-Dome | Bayer AG, Leverkusen, |
| (5-(3,3-dimethyl-1-triazeno)-imidazole-4-carboxamide | | Germany |
| (DTIC)) | | |
| Dactinomycin, actinomycin D | Cosmegen | Merck |
| (actinomycin produced by Streptomyces parvullus, | | |
| C₆₂H₈₆N₁₂O₁₆) | | |
| Darbepoetin alfa | Aranesp | Amgen, Inc., Thousand Oaks, |
| (recombinant peptide) | | CA |
| daunorubicin liposomal | DanuoXome | Nexstar Pharmaceuticals, Inc., |
| ((8S-cis)-8-acetyl-10-[(3-amino-2,3,6-trideoxy-á-L-lyxo- | | Boulder, CO |
| hexopyranosyl)oxy]-7,8,9,10-tetrahydro-6,8,11- | | |
| trihydroxy-1-methoxy-5,12-naphthacenedione | | |
| hydrochloride) | | |
| Daunorubicin HCl, daunomycin | Cerubidine | Wyeth Ayerst, Madison, NJ |
| ((1S,3S)-3-Acetyl-1,2,3,4,6,11-hexahydro-3,5,12- | | |
| trihydroxy-10-methoxy-6,11-dioxo-1-naphthacenyl 3- | | |
| amino-2,3,6-trideoxy-(alpha)-L-lyxo-hexopyranoside | | |
| hydrochloride) | | |
| Denileukin diftitox | Ontak | Seragen, Inc., Hopkinton, MA |
| (recombinant peptide) | | |
| Dexrazoxane | Zinecard | Pharmacia & Upjohn |
| ((S)-4,4′-(1-methyl-1,2-ethanediyl)bis-2,6- | | Company |
| piperazinedione) | | |
| Docetaxel | Taxotere | Aventis Pharmaceuticals, Inc., |
| ((2R,3S)-N-carboxy-3-phenylisoserine, N-tert-butyl ester, | | Bridgewater, NJ |
| 13-ester with 5b-20-epoxy-12a,4,7b,10b,13a- | | |
| hexahydroxytax-11-en-9-one 4-acetate 2-benzoate, | | |
| trihydrate) | | |
| Doxorubicin HCl | Adriamycin, | Pharmacia & Upjohn |
| (8S,10S)-10-[(3-amino-2,3,6-trideoxy-a-L-lyxo- | Rubex | Company |
| hexopyranosyl)oxy]-8-glycolyl-7,8,9,10-tetrahydro- | | |
| 6,8,11-trihydroxy-1-methoxy-5,12-naphthacenedione | | |
| hydrochloride) | | |
| doxorubicin | Adriamycin | Pharmacia & Upjohn |
| | PFS | Company |
| | Intravenous | |
| | injection | |
| doxorubicin liposomal | Doxil | Sequus Pharmaceuticals, Inc., |
| | | Menlo park, CA |
| dromostanolone propionate | Dromostanolone | Eli Lilly & Company, |
| (17b-Hydroxy-2a-methyl-5a-androstan-3-one propionate) | | Indianapolis, IN |
| dromostanolone propionate | Masterone | Syntex, Corp., Palo Alto, CA |
| | injection | |
| Elliott's B Solution | Elliott's B | Orphan Medical, Inc |
| | Solution | |
| Epirubicin | Ellence | Pharmacia & Upjohn |
| ((8S-cis)-10-[(3-amino-2,3,6-trideoxy-a-L-arabino- | | Company |
| hexopyranosyl)oxy]-7,8,9,10-tetrahydro-6,8,11- | | |
| trihydroxy-8-(hydroxyacetyl)-1-methoxy-5,12- | | |
| naphthacenedione hydrochloride) | | |
| Epoetin alfa | Epogen | Amgen, Inc |
| (recombinant peptide) | | |
| Estramustine | Emcyt | Pharmacia & Upjohn |
| (estra-1,3,5(10)-triene-3,17-diol(17(beta))-, 3-[bis(2- | | Company |
| chloroethyl)carbamate] 17-(dihydrogen phosphate), | | |
| disodium salt, monohydrate, or estradiol 3-[bis(2- | | |
| chloroethyl)carbamate] 17-(dihydrogen phosphate), | | |
| disodium salt, monohydrate) | | |
| Etoposide phosphate | Etopophos | Bristol-Myers Squibb |
| (4′-Demethylepipodophyllotoxin 9-[4,6-O-(R)- | | |
| ethylidene-(beta)-D-glucopyranoside], 4′-(dihydrogen | | |
| phosphate)) | | |
| etoposide, VP-16 | Vepesid | Bristol-Myers Squibb |
| (4′-demethylepipodophyllotoxin 9-[4,6-0-(R)-ethylidene- | | |
| (beta)-D-glucopyranoside]) | | |
| Exemestane | Aromasin | Pharmacia & Upjohn |
| (6-methylenandrosta-1,4-diene-3,17-dione) | | Company |
| Filgrastim | Neupogen | Amgen, Inc |
| (r-metHuG-CSF) | | |
| floxuridine (intraarterial) | FUDR | Roche |
| (2′-deoxy-5-fluorouridine) | | |
| Fludarabine | Fludara | Berlex Laboratories, Inc., |
| (fluorinated nucleotide analog of the antiviral agent | | Cedar Knolls, NJ |
| vidarabine, 9-b-D-arabinofuranosyladenine (ara-A)) | | |
| Fluorouracil, 5-FU | Adrucil | ICN Pharmaceuticals, Inc., |
| (5-fluoro-2,4(1H,3H)-pyrimidinedione) | | Humacao, Puerto Rico |
| Fulvestrant | Faslodex | IPR Pharmaceuticals, |
| (7-alpha-[9-(4,4,5,5,5-penta fluoropentylsulphinyl) | | Guayama, Puerto Rico |
| nonyl]estra-1,3,5-(10)-triene-3,17-beta-diol) | | |
| Gemcitabine | Gemzar | Eli Lilly |
| (2′-deoxy-2′,2′-difluorocytidine monohydrochloride (b- | | |
| isomer)) | | |
| Gemtuzumab Ozogamicin | Mylotarg | Wyeth Ayerst |
| (anti-CD33 hP67.6) | | |
| Goserelin acetate | Zoladex | AstraZeneca Pharmaceuticals |
| (acetate salt of [D-Ser(But)⁶,Azgly¹⁰]LHRH; pyro-Glu- | Implant | |
| His-Trp-Ser-Tyr-D-Ser(But)-Leu-Arg-Pro-Azgly-NH2 | | |
| acetate [C₅₉H₈₄N₁₈O₁₄•(C₂H₄O₂)ₓ | | |
| Hydroxyurea | Hydrea | Bristol-Myers Squibb |
| Ibritumomab Tiuxetan | Zevalin | Biogen IDEC, Inc., |
| (immunoconjugate resulting from a thiourea covalent | | Cambridge MA |
| bond between the monoclonal antibody Ibritumomab and | | |
| the linker-chelator tiuxetan [N-[2- | | |
| bis(carboxymethyl)amino]-3-(p-isothiocyanatophenyl)- | | |
| propyl]-[N-[2-bis(carboxymethyl)amino]-2-(methyl)- | | |
| ethyl]glycine) | | |
| Idarubicin | Idamycin | Pharmacia & Upjohn |
| (5,12-Naphthacenedione, 9-acetyl-7-[(3-amino-2,3,6- | | Company |
| trideoxy-(alpha)-L-lyxo-hexopyranosyl)oxy]-7,8,9,10- | | |
| tetrahydro-6,9,11-trihydroxyhydrochloride, (7S-cis)) | | |
| Ifosfamide | IFEX | Bristol-Myers Squibb |
| (3-(2-chloroethyl)-2-[(2-chloroethyl)amino]tetrahydro- | | |
| 2H-1,3,2-oxazaphosphorine 2-oxide) | | |
| Imatinib Mesilate | Gleevec | Novartis AG, Basel, |
| (4-[(4-Methyl-1-piperazinyl)methyl]-N-[4-methyl-3-[[4- | | Switzerland |
| (3-pyridinyl)-2-pyrimidinyl]amino]-phenyl]benzamide | | |
| methanesulfonate) | | |
| Interferon alfa-2a | Roferon-A | Hoffmann-La Roche, Inc., |
| (recombinant peptide) | | Nutley, NJ |
| Interferon alfa-2b | Intron A | Schering AG, Berlin, |
| (recombinant peptide) | (Lyophilized | Germany |
| | Betaseron) | |
| Irinotecan HCl | Camptosar | Pharmacia & Upjohn |
| ((4S)-4,11-diethyl-4-hydroxy-9-[(4-piperi- | | Company |
| dinopiperidino)carbonyloxy]-1H-pyrano[3′,4′: 6,7] | | |
| indolizino[1,2-b] quinoline-3,14(4H,12H) dione | | |
| hydrochloride trihydrate) | | |
| Letrozole | Femara | Novartis |
| (4,4′-(1H-1,2,4-Triazol-1-ylmethylene) dibenzonitrile) | | |
| Leucovorin | Wellcovorin, | Immunex, Corp., Seattle, WA |
| (L-Glutamic acid, N[4[[(2amino-5-formyl-1,4,5,6,7,8- | Leucovorin | |
| hexahydro4oxo6-pteridinyl)methyl]amino]benzoyl], | | |
| calcium salt (1:1)) | | |
| Levamisole HCl | Ergamisol | Janssen Research Foundation, |
| ((−)-(S)-2,3,5,6-tetrahydro-6-phenylimidazo [2,1-b] | | Titusville, NJ |
| thiazole monohydrochloride C₁₁H₁₂N₂S•HCl) | | |
| Lomustine | CeeNU | Bristol-Myers Squibb |
| (1-(2-chloro-ethyl)-3-cyclohexyl-1-nitrosourea) | | |
| Meclorethamine, nitrogen mustard | Mustargen | Merck |
| (2-chloro-N-(2-chloroethyl)-N-methylethanamine | | |
| hydrochloride) | | |
| Megestrol acetate | Megace | Bristol-Myers Squibb |
| 17α(acetyloxy)-6-methylpregna-4,6-diene-3,20-dione | | |
| Melphalan, L-PAM | Alkeran | GlaxoSmithKline |
| (4-[bis(2-chloroethyl) amino]-L-phenylalanine) | | |
| Mercaptopurine, 6-MP | Purinethol | GlaxoSmithKline |
| (1,7-dihydro-6 H-purine-6-thione monohydrate) | | |
| Mesna | Mesnex | Asta Medica |
| (sodium 2-mercaptoethane sulfonate) | | |
| Methotrexate | Methotrexate | Lederle Laboratories |
| (N-[4-[[(2,4-diamino-6- | | |
| pteridinyl)methyl]methylamino]benzoyl]-L-glutamic | | |
| acid) | | |
| Methoxsalen | Uvadex | Therakos, Inc., Way Exton, Pa |
| (9-methoxy-7H-furo[3,2-g][1]-benzopyran-7-one) | | |
| Mitomycin C | Mutamycin | Bristol-Myers Squibb |
| mitomycin C | Mitozytrex | SuperGen, Inc., Dublin, CA |
| Mitotane | Lysodren | Bristol-Myers Squibb |
| (1,1-dichloro-2-(o-chlorophenyl)-2-(p-chlorophenyl) | | |
| ethane) | | |
| Mitoxantrone | Novantrone | Immunex Corporation |
| (1,4-dihydroxy-5,8-bis[[2-[(2- | | |
| hydroxyethyl)amino]ethyl]amino]-9,10-anthracenedione | | |
| dihydrochloride) | | |
| Nandrolone phenpropionate | Durabolin-50 | Organon, Inc., West Orange, |
| | | NJ |
| Nofetumomab | Verluma | Boehringer Ingelheim Pharma |
| | | KG, Germany |
| Oprelvekin | Neumega | Genetics Institute, Inc., |
| (IL-11) | | Alexandria, VA |
| Oxaliplatin | Eloxatin | Sanofi Synthelabo, Inc., NY, NY |
| (cis-[(1R,2R)-1,2-cyclohexanediamine-N,N′] [oxalato(2-)- | | |
| O,O′] platinum) | | |
| Paclitaxel | TAXOL | Bristol-Myers Squibb |
| (5β,20-Epoxy-1,2a,4,7β,10β,13a-hexahydroxytax-11- | | |
| en-9-one 4,10-diacetate 2-benzoate 13-ester with (2R,3S)- | | |
| N-benzoyl-3-phenylisoserine) | | |
| Pamidronate | Aredia | Novartis |
| (phosphonic acid (3-amino-1-hydroxypropylidene) bis-, | | |
| disodium salt, pentahydrate, (APD)) | | |
| Pegademase | Adagen | Enzon Pharmaceuticals, Inc., |
| ((monomethoxypolyethylene glycol succinimidyl) 11-17- | (Pegademase | Bridgewater, NJ |
| adenosine deaminase) | Bovine) | |
| Pegaspargase | Oncaspar | Enzon |
| (monomethoxypolyethylene glycol succinimidyl | | |
| L-asparaginase) | | |
| Pegfilgrastim | Neulasta | Amgen, Inc |
| (covalent conjugate of recombinant methionyl human G- | | |
| CSF (Filgrastim) and monomethoxypolyethylene glycol) | | |
| Pentostatin | Nipent | Parke-Davis Pharmaceutical |
| | | Co., Rockville, MD |
| Pipobroman | Vercyte | Abbott Laboratories, Abbott |
| | | Park, IL |
| Plicamycin, Mithramycin | Mithracin | Pfizer, Inc., NY, NY |
| (antibiotic produced by Streptomyces plicatus) | | |
| Porfimer sodium | Photofrin | QLT Phototherapeutics, Inc., |
| | | Vancouver, Canada |
| Procarbazine | Matulane | Sigma Tau Pharmaceuticals, |
| (N-isopropyl-μ-(2-methylhydrazino)-p-toluamide | | Inc., Gaithersburg, MD |
| monohydrochloride) | | |
| Quinacrine | Atabrine | Abbott Labs |
| (6-chloro-9-(1-methyl-4-diethyl-amine) butylamino-2- | | |
| methoxyacridine) | | |
| Rasburicase | Elitek | Sanofi-Synthelabo, Inc., |
| (recombinant peptide) | | |
| Rituximab | Rituxan | Genentech, Inc., South San |
| (recombinant anti-CD20 antibody) | | Francisco, CA |
| Sargramostim | Prokine | Immunex Corp |
| (recombinant peptide) | | |
| Streptozocin | Zanosar | Pharmacia & Upjohn |
| (streptozocin 2-deoxy-2- | | Company |
| [[(methylnitrosoamino)carbonyl]amino]-a(and b)-D- | | |
| glucopyranose and 220 mg citric acid anhydrous) | | |
| Talc | Sclerosol | Bryan, Corp., Woburn, MA |
| (Mg₃Si₄O₁₀ (OH)₂) | | |
| Tamoxifen | Nolvadex | AstraZeneca Pharmaceuticals |
| ((Z)2-[4-(1,2-diphenyl-1-butenyl) phenoxy]-N,N- | | |
| dimethylethanamine 2-hydroxy-1,2,3- | | |
| propanetricarboxylate (1:1)) | | |
| Temozolomide | Temodar | Schering |
| (3,4-dihydro-3-methyl-4-oxoimidazo[5,1-d]-as-tetrazine- | | |
| 8-carboxamide) | | |
| Teniposide, VM-26 | Vumon | Bristol-Myers Squibb |
| (4′-demethylepipodophyllotoxin 9-[4,6-0-(R)-2- | | |
| thenylidene-(beta)-D-glucopyranoside]) | | |
| Testolactone | Teslac | Bristol-Myers Squibb |
| (13-hydroxy-3-oxo-13,17-secoandrosta-1,4-dien-17-oic | | |
| acid [dgr]-lactone) | | |
| Thioguanine, 6-TG | Thioguanine | GlaxoSmithKline |
| (2-amino-1,7-dihydro-6 H - purine-6-thione) | | |
| Thiotepa | Thioplex | Immunex Corporation |
| (Aziridine,1,1′,1″-phosphinothioylidynetris-, or Tris (1- | | |
| aziridinyl) phosphine sulfide) | | |
| Topotecan HCl | Hycamtin | GlaxoSmithKline |
| ((S)-10-[(dimethylamino) methyl]-4-ethyl-4,9-dihydroxy- | | |
| 1H-pyrano[3′,4′: 6,7] indolizino [1,2-b] quinoline-3,14- | | |
| 4H,12H)-dione monohydrochloride) | | |
| Toremifene | Fareston | Roberts Pharmaceutical Corp., |
| (2-(p-[(Z)-4-chloro-1,2-diphenyl-1-butenyl]-phenoxy)- | | Eatontown, NJ |
| N,N-dimethylethylamine citrate (1:1)) | | |
| Tositumomab, I 131 Tositumomab | Bexxar | Corixa Corp., Seattle, WA |
| (recombinant murine immunotherapeutic monoclonal | | |
| IgG₂ₐ lambda anti-CD20 antibody (I 131 is a | | |
| radioimmunotherapeutic antibody)) | | |
| Trastuzumab | Herceptin | Genentech, Inc |
| (recombinant monoclonal IgG₁ kappa anti-HER2 | | |
| antibody) | | |
| Tretinoin, ATRA | Vesanoid | Roche |
| (all-trans retinoic acid) | | |
| Uracil Mustard | Uracil | Roberts Labs |
| | Mustard | |
| | Capsules | |
| Valrubicin, N-trifluoroacetyladriamycin-14-valerate | Valstar | Anthra --> Medeva |
| ((2S-cis)-2-[1,2,3,4,6,11-hexahydro-2,5,12-trihydroxy-7 | | |
| methoxy-6,11-dioxo-[[4 2,3,6-trideoxy-3- | | |
| [(trifluoroacetyl)-amino-α-L-lyxo-hexopyranosyl]oxyl]-2- | | |
| naphthacenyl]-2-oxoethyl pentanoate) | | |
| Vinblastine, Leurocristine | Velban | Eli Lilly |
| (C₄₆H₅₆N₄O₁₀•H₂SO₄) | | |
| Vincristine | Oncovin | Eli Lilly |
| (C₄₆H₅₆N₄O₁₀•H₂SO₄) | | |
| Vinorelbine | Navelbine | GlaxoSmithKline |
| (3′,4′-didehydro-4′-deoxy-C′-norvincaleukoblastine [R- | | |
| (R*,R*)-2,3-dihydroxybutanedioate (1:2)(salt)]) | | |
| Zoledronate, Zoledronic acid | Zometa | Novartis |
| ((1-Hydroxy-2-imidazol-1-yl-phosphonoethyl) | | |
| phosphonic acid monohydrate) | | |

III. Drug Screening Applications

In some embodiments, the present invention provides drug screening assays (e.g., to screen for anticancer drugs). The screening methods of the present invention utilize cancer markers identified using the methods of the present invention (e.g., including but not limited to, SPINK1 and/or EGFR). For example, in some embodiments, the present invention provides methods of screening for compounds that alter (e.g., decrease) the expression of SPINK1 and/or EGFR. The compounds or agents may interfere with transcription, by interacting, for example, with the promoter region. The compounds or agents may interfere with mRNA produced from SPINK1 and/or EGFR (e.g., by RNA interference, antisense technologies, etc.). The compounds or agents may interfere with pathways that are upstream or downstream of the biological activity of SPINK1 and/or EGFR. In some embodiments, candidate compounds are antisense or interfering RNA agents (e.g., oligonucleotides) directed against cancer markers. In other embodiments, candidate compounds are antibodies or small molecules that specifically bind to SPINK1 and/or EGFR and inhibit its biological function.

In one screening method, candidate compounds are evaluated for their ability to alter SPINK1 and/or EGFR expression by contacting a compound with a cell expressing SPINK1 and/or EGFR and then assaying for the effect of the candidate compounds on expression. In some embodiments, the effect of candidate compounds on expression of SPINK1 and/or EGFR is assayed for by detecting the level of SPINK1 mRNA expressed by the cell. mRNA expression can be detected by any suitable method.

In other embodiments, the effect of candidate compounds on expression of SPINK1 and/or EGFR is assayed by measuring the level of polypeptide encoded by SPINK1 and/or EGFR. The level of polypeptide expressed can be measured using any suitable method, including but not limited to, those disclosed herein.

Specifically, the present invention provides screening methods for identifying modulators, i.e., candidate or test compounds or agents (e.g., antibodies, proteins, peptides, peptidomimetics, peptoids, small molecules or other drugs) which bind to SPINK1 and/or EGFR, have an inhibitory (or stimulatory) effect on, for example, SPINK1 and/or EGFR expression or activity, or have a stimulatory or inhibitory effect on, for example, the expression or activity of a SPINK1 and/or EGFR substrate. Compounds thus identified can be used to modulate the activity of target gene products (e.g., SPINK1 and/or EGFR) either directly or indirectly in a therapeutic protocol, to elaborate the biological function of the target gene product, or to identify compounds that disrupt normal target gene interactions. Compounds that inhibit the activity or expression of cancer markers are useful in the treatment of proliferative disorders, e.g., cancer, particularly prostate cancer.

In one embodiment, the invention provides assays for screening candidate or test compounds that are substrates of SPINK1 and/or EGFR protein or polypeptide or a biologically active portion thereof. In another embodiment, the invention provides assays for screening candidate or test compounds that bind to or modulate the activity of a SPINK1 and/or EGFR protein or polypeptide or a biologically active portion thereof.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. NatI. Acad. Sci. 87:6378-6382 [1990]; Felici, J. Mol. Biol. 222:301 [1991]).

### Experimental

The following examples are provided in order to demonstrate and further illustrate certain preferred embodiments and aspects of the present invention and are not to be construed as limiting the scope thereof.

### EXAMPLE 1

### Material and Methods

### Cell Lines and SPINK1 Knockdown

The benign immortalized prostate cell line RWPE, prostate cancer cell lines DU145, PC3 and 22RV1 were obtained from the American Type Culture Collection (ATCC, Manassas, Va.) and were grown according to ATCC guidelines. For stable knockdown of SPINK1, human lentiviral shRNAmir individual clone (ID V2LHS_{—}153419) targeting against SPINK1 or non-silencing lentiviral shRNAmir in GIPZ vectors were purchased from Open Biosystems (Thermo Scientific Open Biosystems, Hunsville, Ala.). Lentiviruses from these constructs were generated by the University of Michigan Vector Core. Viral particle infections were carried out in the presence of polybrene (8 μg/ml) in 50-60% confluent 22RV1 cells. After 48 hours, infected cells were grown in 22RV1 culture media containing puromycin (2 μg/ml). Three weeks later, stable cells were plated into 96 well plates for clonal selection. SPINK1 knockdown was confirmed in pooled and single clones by qPCR and single clones showing highest knockdown were further expanded. For siRNA mediated knockdowns, the most effective siRNA duplex against SPINK1 (J-019724-07; Dharmacon, Chicago) EGFR (J-003114-13; Dharmacon) or siCONTROL Non-Targeting siRNA #1 (D-001210-01) was used. All transfections were carried out in the presence of Oligofectamine (Invitrogen), according to manufacturer's instructions. After 24 hr, a second identical transfection was carried out, and cells were harvested 24 hr later for RNA isolation, invasion assays, or proliferation assays. All transient or stable 22RV1 cells were tested for SPINK1 knockdown by qPCR. Sequences of siRNAs are as follows:

| | |
|---|---|
| J-019724-05: GGAAAUACUUAUCCCAAUG | (SEQ ID NO: 5) |
| J-019724-06: UAAUGGAUGCACCAAGAUA | (SEQ ID NO: 6) |
| J-019724-07: GAAGAGAGGCCAAAUGUUA | (SEQ ID NO: 7) |
| J-003114-13: CAGAGGAUGUUCAAUAACU | (SEQ ID NO: 12) |

### Quantitative PCR (QPCR)

Total RNA was isolated using miRNeasy mini kit following manufacturer's instruction (Qiagen). Complimentary DNA was synthesized from one microgram of total RNA, using SuperScript III (Invitrogen) in the presence of random primers. The reaction was carried out for 60 minutes at 50° C. and the cDNA was purified using microcon YM-30 (Millipore Corp, Bedford, Mass., USA) according to manufacturer's instruction and used as template in quantitative PCRs. All oligonucleotide primers used in this study were synthesized by Integrated DNA Technologies (Coralville, Iowa). Quantitative PCR (qPCR) was performed using the StepOne Real Time PCR system (Applied Biosystems, Foster City, Calif.). Briefly, reactions were performed with SYBR Green Master Mix (Applied Biosystems) and 25 ng of both the forward and reverse primers for SPINK1 (TGTCTGTGGGACTGATGGAA (SEQ ID NO:1)) and AGGCCCAGATTTTTGAATGA (SEQ ID NO:2), PRSS1 (5′-GCCTGGACGCTCCTGTGCTG-3 (SEQ ID NO:8)′ and 5′-CTGGGCACAGCCATCACCCC-3′ (SEQ ID NO:9)) and EGFR (5′-GGGCCAGGTCTTGAAGGCTGT-3′ (SEQ ID NO:10) and 5′-ATCCCCAGGGCCACCACCAG-3′ (SEQ ID NO:11)) using the manufacturer recommended thermocycling conditions. For each experiment, threshold levels were set during the exponential phase of the qPCR reaction using the StepOne software. The amount of each target gene relative to the housekeeping gene glyceraldehyde-3-phosphate dehydrogenase (GAPDH; forward TGCACCACCAACTGCTTAGC (SEQ ID NO:3) and reverse primers GGCATGGACTGTGGTCATGAG (SEQ ID NO:4)) for each sample was determined using the comparative threshold cycle (Ct) method. Threshold levels for each experiment were set during the exponential phase of the QPCR reaction using Sequence Detection Software version 1.2.2 (Applied Biosystems).

### Cell Proliferation Assay

Proliferation for control and experimental cells was measured by a colorimetric assay based on the cleavage of the tetrazolium salt WST-1 by mitochondrial dehydrogenases (cell proliferation reagent WST1; Roche Diagnostics, Mannheim, Germany) at the indicated time points in triplicate. Cell counts for shNS vector and shSPINK1 cells were estimated by trypsinizing cells and analysis by Coulter counter (Beckman Coulter, Fullerton, Calif., USA) at different time points in triplicates.

### Soft Agar Colony Assay

A 50 μL base layer of agar (0.6% Agar in DMEM with 10% FBS) was allowed to solidify in a 96-well flat-bottom plate prior to the addition of 754, stable shNS vector and shSPINK1 cell suspension containing 4,000 cells in 0.4% Agar in DMEM with 10% FBS. The cell containing layer was then solidified at 4° C. for 15 minutes prior to the addition of 1004, of MEM with 5% FBS. Colonies were allowed to grow for 21 days before imaging under a light microscope.

Cell Motility Assay

22RV1 or stable shNS vector and shSPINK1 cells were plated on a lawn of microscopic fluorescent beads on collagen coated 96-well plates (Cellomics, Thermo Scientific, Pa., US). Motile cells push the beads and create phagokinetic tracks behind each cell. The cleared track area is proportional to the magnitude of cell motility. Plates were analyzed and images were captured using standard light microscopy.

### Basement Membrane Matrix Invasion Assay

For invasion assays, shNS vector or shSPINK1 cells, RWPE, PC3 and 22RV1 cells were used. Equal numbers of the indicated cells were seeded onto the basement membrane matrix (EC matrix; Chemicon, Temecula, Calif., USA) present in the insert of a 24-well culture plate, with fetal bovine serum added to the lower chamber as a chemoattractant. After 48 hr, non-invading cells and EC matrix were removed using a cotton swab. Invaded cells were stained with crystal violet and photographed. The inserts were treated with 10% acetic acid, and absorbance was measured at 560 nm.

### 22RV1 or SPINK1 Knockdown Xenograft Models

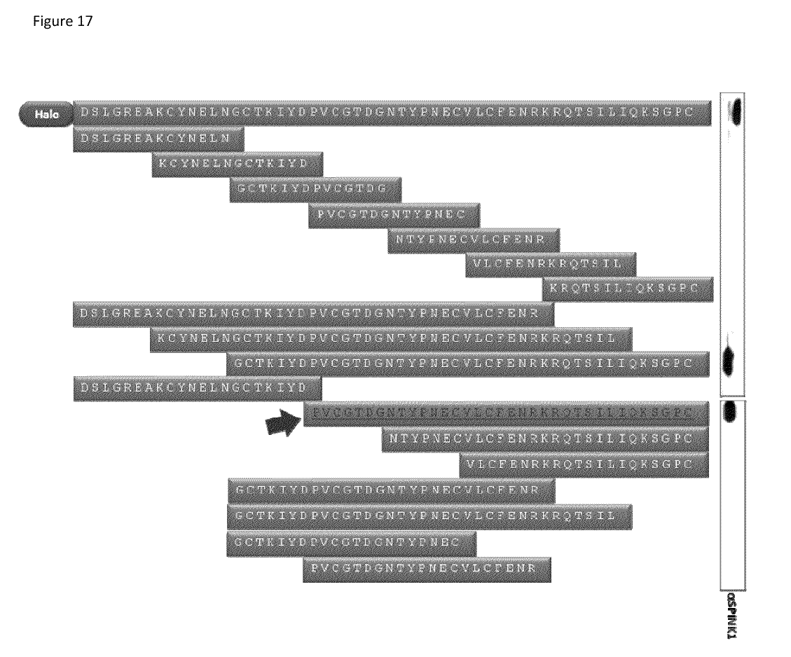

Four weeks old male Balb/C nu/nu mice were purchased from Charles River, Inc. (Charles River Laboratory, Wilmington, Mass.). Stable shNS-luciferase and shSPINK1-luciferase cells (5×10⁵ cells) or 22RV1-Luc (2×10⁵ cells) or PC-3 luciferase cells were resuspended in 100 μl of saline with 20% Matrigel (BD Biosciences, Becton Drive, N.J.) and were implanted subcutaneously into the left flank regions of the mice. Mice were anesthetized using a cocktail of xylazine (80-120 mg/kg IP) and ketamine (10 mg/kg IP) for chemical restraint before implantation. Eight mice were included in each group. Mice implanted with 22RV1-luciferase or PC-3 luciferase cells were randomly divided into two groups, and treated twice a week with SPINK1 mAb (Mobitec Inc; Goettingen, Germany) or control mouse IgG antibody (Millipore; Kankakee, Ill.) at the dose of 10 mg/Kg body weight. Epitope mapping of the SPINK1 mAb is shown in FIG. 17. Growth in tumor volume was recorded weekly by using digital calipers and tumor volumes were calculated using the formula (π/6) (L×W2), where L=length of tumor and W=width. Antitumor activity was determined from the analyses of tumor growth inhibition, defined as the decrease in the mean tumor volume for SPINK1 mAb treated mice versus mouse IgG mAb treated mice. In vivo bioluminescent imaging was performed weekly upto week 4 using IVIS-200 imaging system (Xenogen Corp.). Mice were injected 150 mg/kg luciferin intra-peritoneal 12 min before imaging. All images were collected and analyzed with Living Image software (Xenogen Corp.). All procedures involving mice were approved by the University Committee on Use and Care of Animals (UCUCA) at the University of Michigan and conform to their relevant regulatory standards.

### Immunohistochemistry and Immunofluorescence Staining

FFPE sections were obtained from formalin-fixed xenografted tumors and, antigen retrieval was performed by microwaving sections in citrate buffer (pH6.0) for 10 min, followed by cooling and rinse in water. Sections were further blocked in hydrogen peroxidase for 5 min followed by incubation in anti-Ki-67 antibody at 1:400 dilutions (AbCam, Cat#ab15580) for 30 min at room temperature. Sections were further incubated in EnVision+ for 30 min at room temperature; immunoreactive nuclei were visualized with the Vectastain Elite ABC Kit using diaminobenzidine (DAB) as the substrate (Vector Laboratories, Inc.). Finally, sections were counterstained with Harris Hematoxlyin (Fisher), dehydrated, and mounted with Permount (Fisher). Immunoreactive positive Ki-67 nuclei were scored blindly for both groups in 400× magnification.

For immunofluorescence staining, shNS-luciferase and shSPINK1-luciferase cells were grown in chamber slides at sub-confluent density. Cells were fixed using chilled methanol after washing with 1XPBS. The chamber slides containing cells were then blocked in PBS-T containing 5% normal donkey serum for 1 hour at room temperature. Slides were incubated overnight at 4° C. with a mouse anti-SPINK1 antibody (H00006690-M01; Abnova, Taipei City, Taiwan) 1:10,000 dilution, then washed, and followed by secondary antibodies (anti-mouse Alexa 555 1:1000 dilutions) for 1 hour. Slides were mounted using Vectashield mounting medium containing DAPI (Vector Laboratories, Burlingame, Calif.) after washing with PBS-T and PBS. Fluorescence images were captured using a Zeiss Microscope (Carl Zeiss, Gottingen, Germany) equipped with a high resolution CCD camera controlled by ISIS image processing software (Metasystems, Germany).

### Statistical Analysis

All values presented in the study were expressed as mean±SEM. The significant differences between the groups were analyzed by a Student's t test and a P value of <0.05 was considered significant.

### Production of Recombinant SPINK1 Protein

A synthetic construct with SPINK1-2xV5 cloned into pcDNA3.1 using KpnI and XhoI restriction sites was purchased from GENEART (Regensburg, Germany). The full length SPINK1 cDNA including signal peptide was amplified and subcloned downstream of the Met-Arg-Gly-Ser-His₆ (MRGSH₆) tag coding sequence into the BamHI/PstI restriction enzyme sites of the pQE-9 expression vector (Qiagen, Hilden, Germany). The plasmid was sequenced to verify integrity. Production of the recombinant protein from pQE-9 plasmids was carried out using M15 cells treated with isopropyl-1-thio-D-galactopyranoside (400 mM). Bacterial cell lysates were centrifuged and the supernatants were purified by affinity chromatography using a Co²⁺-agarose resin (Clontech, Saint-Germain-en-Laye, France). Multiple tags protein including 6× His protein (GenScript Corp., Piscataway, N.J.) was used as a control. Bacterially expressed recombinant SPINK1 protein was separated on 5-30% sodium dodecyl sulfate-polyacrylamide gels under reducing conditions. In order to get concentrated SPINK1 fraction from CM of 22RV1 cells or control CM form RWPE cells, proteins were separated by ultrafiltration according to their molecular weight (MW), using membranes at a cutoff of 3-10 kDa (SPINK1 molecular weight 6.2 kDa). Protein was transferred onto Polyvinylidene Difluoride membrane (GE Healthcare) and membrane was incubated for one hour in blocking buffer [Tris-buffered saline, 0.1% Tween (TBS-T), 5% nonfat dry milk] and probed with the SPINK1 mAb (Mobitec Inc; Goettingen, Germany). After washing the blots with TBS-T, the blots were incubated with horseradish peroxidase-conjugated secondary mouse antibody and the signals visualized by enhanced chemiluminescence system as described by the manufacturer (GE Healthcare).

### Immunoprecipitation and Western Blot Analysis

Briefly, transiently EGFR expressing HEK293 cells were washed twice PBS supplemented with protease inhibitor. Cells were lysed in Triton X-100 lysis buffer (20 mM MOPS, pH 7.0, 2 mM EGTA, 5 mM EDTA, 30 mM sodium fluoride, 60 mM β-glycerophosphate, 20 mM sodium pyrophosphate, 1 mM sodium orthovanadate, 1% Triton X-100, protease inhibitor cocktail (Roche). Cell lysates (0.5-1.0 mg) were then pre-cleaned with protein A/G agarose beads (Santa Cruz) by incubation for 1 hour with shaking at room temperature followed by centrifugation at 2000× g for 1 minute. Recombinant SPINK1-GST (Proteintech Group Inc.), GST (AbCam) or GST-VEGF Receptor 2 protein (Cell Signaling) (80 μg/ml) were added to the pre-cleaned protein lysates and incubated at 4° C. overnight. Similarly, 22Rv1 cells lysate in Kinet lysis buffer was mixed with SPINK1-GST recombinant protein (80 m/ml) and incubated at 4° C. overnight. After adding 2 μg of each antibody (mouse IgG, Millipore; SPINK1 mAb, Mobitec Inc.; EGFR mAb, Cell Signaling) lysates were further incubated with shaking at 4° C. for 4 hours prior to addition of 20 μL protein A/G agarose beads (Santa Cruz). The mixture was then incubated with shaking at 4° C. for another 4 hours prior to washing the lysate-bead precipitate (centrifugation at 2000×g for 1 minute) 3 times in Triton X-100 lysis buffer or Kinet lysis buffer.

Beads were finally precipitated by centrifugation, resuspended in 254, of 2× loading buffer and boiled at 80° C. for 10 minutes. Samples were then analyzed by SDS-PAGE Western blot analysis as described below.

### Western Blot Analysis

Cell lysates were prepared in RIPA lysis buffer (Thermo Scientific), supplemented with complete proteinase inhibitor and phosphatase inhibitor mixture (Roche). Fifteen micrograms of each protein extract was boiled in sample buffer, separated by SDS-PAGE, and transferred onto Polyvinylidene Difluoride membrane (GE Healthcare). rSPINK1 stimulated 22RV1 phospho-EGFR blot was performed as described before (Jemal et al., 2010. CA Cancer J Clin 60, 277-300 (2010)). The membrane was incubated for one hour in blocking buffer (Tris-buffered saline, 0.1% Tween (TBS-T), 5% nonfat dry milk) and incubated overnight at 4° C. with anti-phospho-MEK or -ERK or -EGFR or -AKT antibodies or total -MEK or -ERK or -AKT and -EGFR antibodies (Cell Signaling); trypsinl polyclonal antibody (Abcam) and PSA monoclonal antibody (Dako). Following three washes with TBS-T, the blot was incubated with horseradish peroxidase-conjugated secondary antibody and the signals visualized by enhanced chemiluminescence system as described by the manufacturer (GE Healthcare).

### EGFR Cross Linking Immunoblotting

22RV1 cells were stimulated with rSPINK1 (100 ng/ml) or EGF (10 ng/ml) in 6-well plates for 0, 5, 10, 30, 60 and 90 min at 37° C. At the end of each time point cells were washed twice with 1× PBS. Cells were then treated with cross linking reagent BS3 [Bis-(sulfosuccinimidyl) suberate] to a final concentration of 5 mM and incubated on ice for 2 h. The quench solution (1M Tris, pH 7.5, 1:100 dilutions) was then added to a final concentration of 10 mM and incubated for 15 min on ice. The cells were then lysed with RIPA buffer supplemented with protease inhibitor and phosphatase inhibitors (Roche). EGFR dimerization was analyzed by Non-reducing immunoblot.

### Serum Toxicity Marker Analyses

At the end of the xenograft study, mice were anaesthetized and blood was collected by cardiac puncture. Blood was transferred into a 1.5 ml eppendorf tube and kept on ice for 45 min, followed by centrifugation at 8000 rpm for 10 min at 4° C. Clear supernatant containing serum was collected and transferred into a sterile 1.5 ml eppendorf tube. All serum markers were measured using dry-slide technology on IDEXX Vettest 8008 biochemical analyser (IDEXX, France). About 50 μL of the serum sample was loaded on the VetTest pipette tip followed by securely fitting it on the pipettor and manufacturer's instructions were followed for further analyses.

### Chick Chorioallantoic Membrane (CAM) Assay

The assay was performed essentially as described (Zijlstra et al., Cancer Res 62, 7083-7092 (2002)). Two million RWPE cells were mixed with either 200 ng multiple tag control protein or 200 ng of rSPINK1 protein and applied to the chorioallantoic membrane (CAM) of 11-day old chicken embryo. Similarly, two million 22RV1 or PC3 cells were mixed with either monoclonal IgG or anti-SPINK1 or C225 (1 μg/ml) and applied onto the upper CAM of a fertilized chicken embryo. Three days post-implantation, the relative number of cells that intravasate into the vasculature of the lower CAM was analyzed by extracting genomic DNA using the Purgene DNA purification system. Quantification of the human cells in the extracted DNA was done as described (van der Horst et al., Biotechniques 37, 940-942, 944, 946 (2004)).

### Results

### SPINK1 as an Autocrine Factor in Prostate Cancer

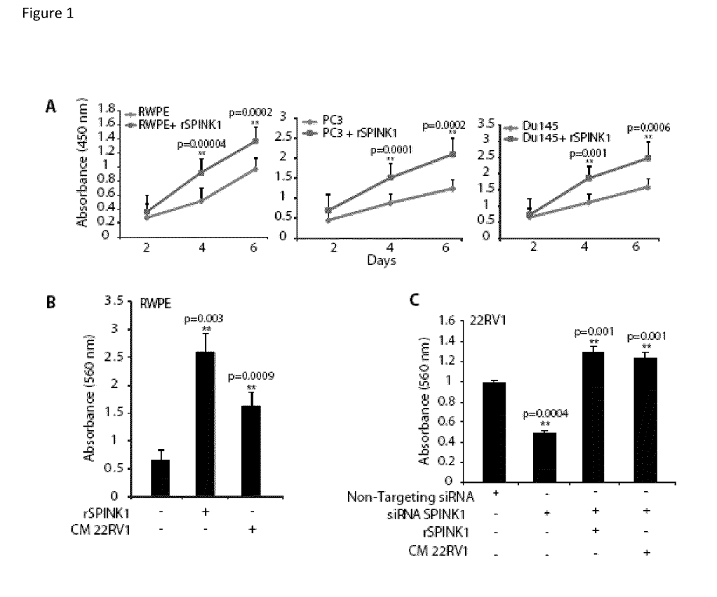

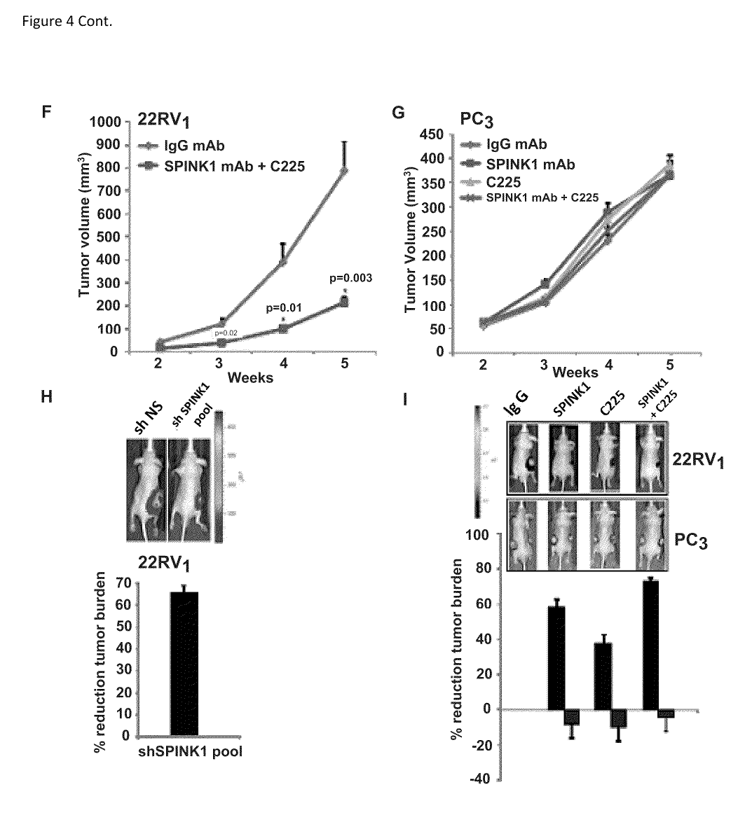

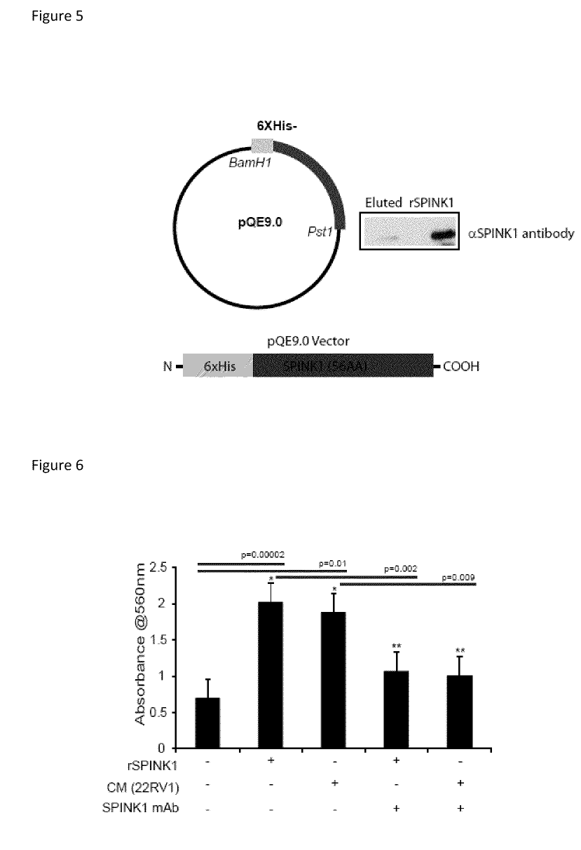

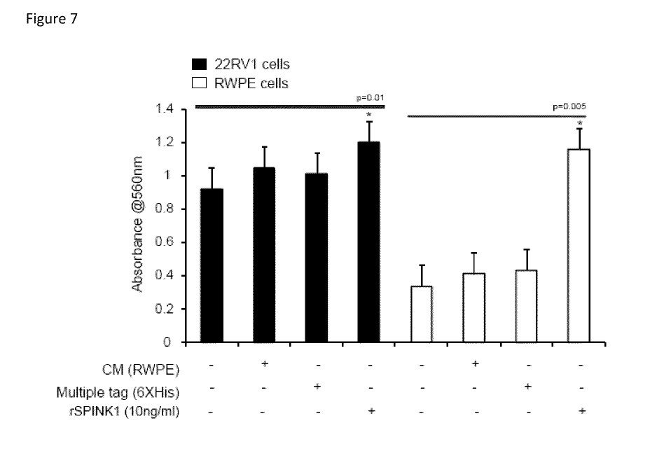

This Example describes the investigation of the role of autocrine SPINK1 in invasion and proliferation using recombinant 6× His-tagged SPINK1 protein (rSPINK1) (FIG. 5) or conditioned media (CM) collected from 22RV1 prostae cancer cells. Benign immortalized RWPE prostate epithelial cells and DU145 and PC3 prostate cancer cells (both SPINK1⁻/ETS⁻) were treated with rSPINK1 (10 ng/ml). This resulted in a significant increase in cell proliferation compared to controls over a six day time course (FIG. 1A). The effect of rSPINK1 or CM on cell invasion was next characterized using a Boyden chamber Matrigel invasion assay. As shown in FIG. 1B, addition of rSPINK1 or CM to RWPE cells significantly increased invasion (P=0.003, P=0.0009 respectively), which was attenuated (P=0.008) by neutralizing with SPINK1 mAb. Similar effects were observed when a breast cancer MCF7 cells were treated with rSPINK1 or CM either in the presence or absence of anti-SPINK1 mAb (FIG. 6). CM from RWPE cells or multiple 6× His tagged protein controls did not mediate invasion of RWPE or 22RV1 cells (FIG. 7).

It was previously shown that transient siRNA mediated silencing of SPINK1 in 22RV 1 cells decreased cell invasion (Tomlins et al, Cancer Cell 13, 519-528 (2008)). In this study, several siRNA sequences had similar phenotypic effects and the one with the most robust knock-down was identified for subsequent studies. These results were extended by demonstrating that the addition of rSPINK1 or 22RV1 CM to 22RV1 cells treated with siRNA against SPINK1 rescued the invasive phenotype (FIG. 1C, P=0.001 in both cases). Together, these findings show that extracellular SPINK1 induces prostate cancer cell proliferation and invasion, indicating that SPINK1 is an autocrine pro-proliferative and pro-invasive factor.

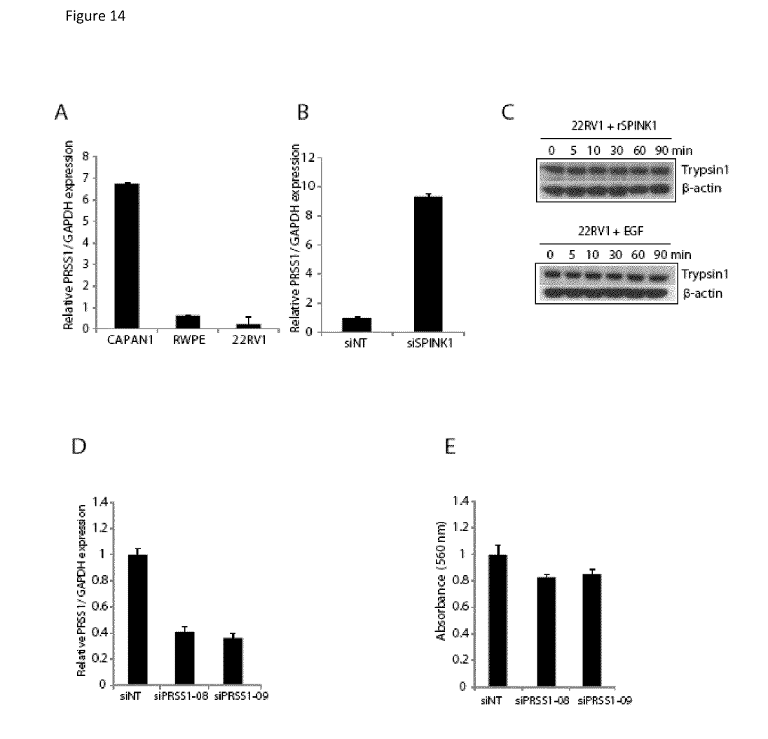

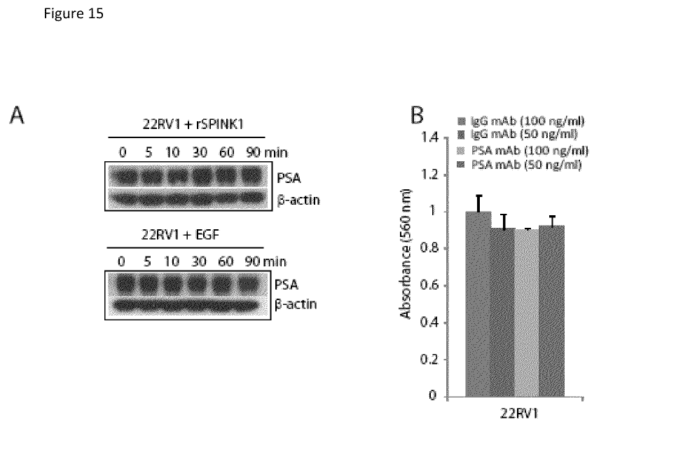

It was next investigated whether the exogenous effect of SPINK1 on cell proliferation and invasion is dependent on protease inhibitory activity of trypsin (which has been shown to be simultaneously expressed with SPINK1 in different tumor types (Hotakainen et al., Int J Oncol 28, 95-101 (2006); Paju et al., Clin Cancer Res 10, 4761-4768 (2004)) or PSA. Experiments demonstrated that PRSS1 (trypsinogen) mRNA expression in 22RV1 cells is relatively low (FIG. 14A), although a significant increase in PRSS1 transcript was observed in siRNA mediated SPINK1 knockdown 22RV1 cells (FIG. 14B). However, as shown in FIG. 14C, stimulation of 22RV1 cells with rSPINK1 or EGF did not affect trypsin expression. siRNA mediated knockdown of PRSS1 in 22RV1 cells also had no effect on invasion (FIGS. 14D & E). Similarly, stimulation of 22RV1 cells with rSPINK1 or EGF did not affect PSA expression (FIG. 15A). Finally, blocking PSA using a monoclonal antibody did not significantly inhibit 22RV1 cell invasion (FIG. 15B). Together, these findings demonstrate that extracellular SPINK1 induces prostate cancer cell proliferation and invasion independent of protease inhibitory activity of trypsin or PSA. The results indicate that SPINK1 is an autocrine pro-proliferative and pro-invasive factor with effects independent of protease activity.

### Role of SPINK1 in Cell Proliferation and Invasion

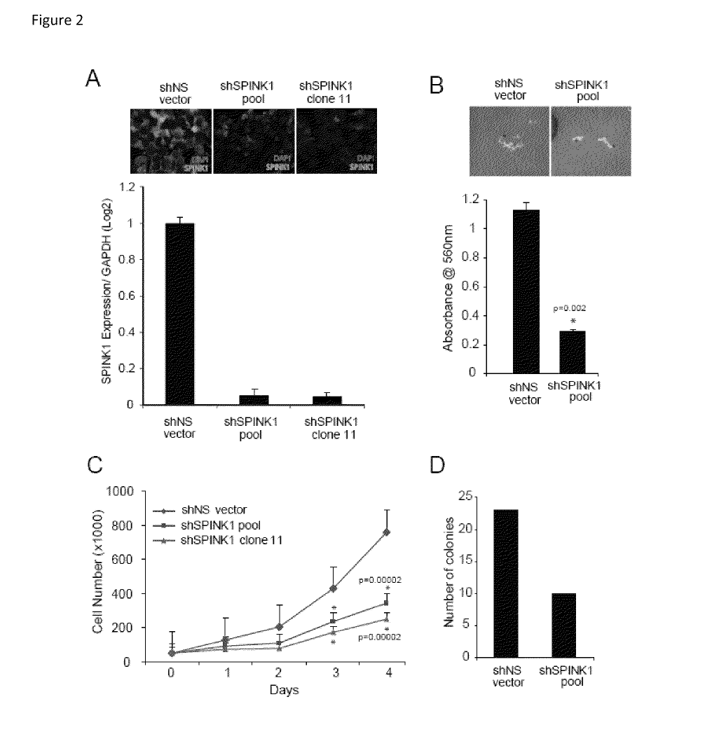

To further investigate the role of SPINK1 in cell proliferation and invasion, shRNA against SPINK1 were generated and stable 22RV1 cells in which SPINK1 was silenced (shSPINK1) were established. Knockdown of SPINK1 in both pooled and clonal shSPINK1 cells was confirmed at the RNA level by quantitative PCR and at the protein level by immunoflourescence staining using an antibody against SPINK1 (FIG. 2A). Next, the effect of SPINK1 on cell invasion and motility was investigated using shSPINK1 cells. shSPINK1 cells showed decreased invasion in a Boyden chamber Matrigel assay compared to non-specific vector control (shNS) cells (FIG. 2B; P=0.002 in both cases). shSPINK1 cells also showed reduced cell motility compared to shNS cells in a bead motility assay (FIG. 2B).

To investigate the role of SPINK1 in cell proliferation, assays were performed using pooled shSPINK1 cells, the clone with the greatest SPINK1 knockdown (shSPINK1 clone 11), and 22RV1 cells with stable knockdown of a non-specific vector control (shNS). Both pooled and single clone of shSPINK1 cells showed a significantly decreased proliferation compared to shNS cells (FIG. 2C; P=0.00002 in both cases). By soft agar colony formation assay, shSPINK1 cells also showed decreased colonies compared to shNS vector cells (FIG. 2D).

### In Vitro Targeting of SPINK1 Using a Monoclonal Antibody

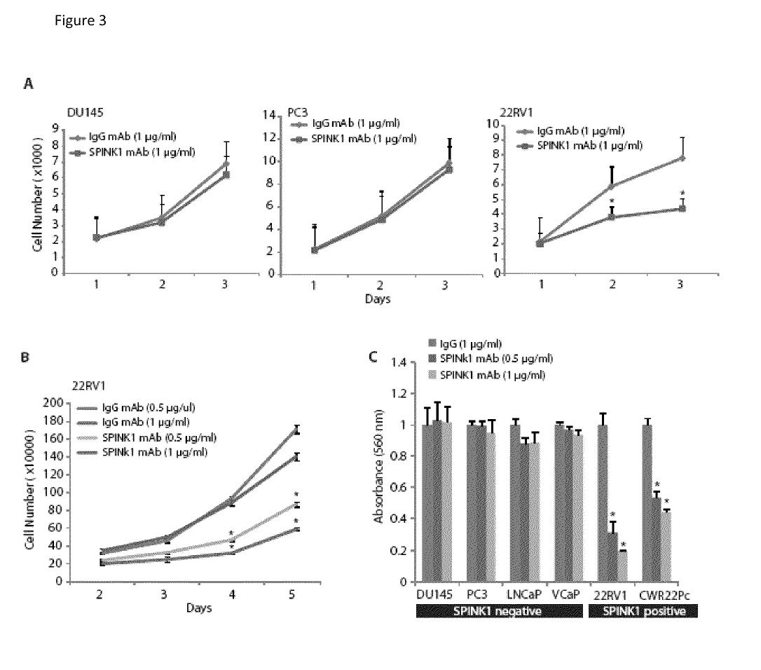

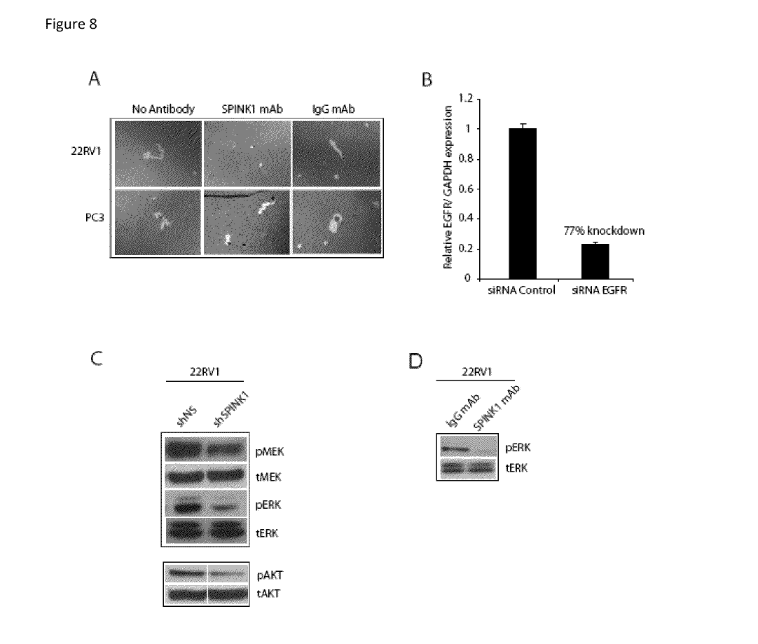

As a monoclonal antibody was able to attenuate the increased cell invasion caused by rSPINK1 in RWPE cells (FIG. 1B), it was contemplated that this antibody may be used to directly target SPINK1⁺/ETS⁻ prostate cancer cells. Thus, the effects of the anti-SPINK1 mAb (Mobitec, Gottingen, Germany) on 22RV1 cell invasion were assayed, and it was found that the anti-SPINK1 mAb (0.5-1 μg/ml) significantly attenuated cell invasion as compared to a control monoclonal IgG antibody (FIG. 3A; P=0.0003 and P=0.0007 respectively). The anti-SPINK1 mAB had no significant effect on PC3 (SPINK1⁻/ETS⁻) prostate cancer cell invasion (FIG. 3B). Similarly, the anti-SPINK1 mAb attenuated 22RV1 cell motility compared to IgG control, but had no effect on DU145 or PC3 cell motility (FIG. 8).

In addition to inhibiting proliferation, anti-SPINK1 mAb (0.5 and 1 μg/ml) significantly attenuated cell invasion by 69% and 81% respectively as compared to a control IgG mAb in 22RV1 cells (FIG. 3C; P=0.002 and P=0.007 respectively). Similar to 22RV1, which is an androgen signaling independent derivative of primary CWR22 human prostate xenograft tumors. CWR22Pc cells, an androgen signaling dependent derivative of CWR22 (Dagvadorj et al., Clin Cancer Res 14, 6062-6072 (2008)), which also express high levels of SPINK1, were also investigaed. CWR22Pc cell invasion was blocked by 47 and 54% by anti-SPINK1 mAb at 0.5 and l μg/ml of SPINK1 mAb concentration (FIG. 3C; P=0.003 and P=0.002 respectively). The anti-SPINK1 mAb had no significant effect on invasion of SPINK1-prostate cancer cell lines including PC3, DU145, LNCaP or VCaP (FIG. 3C). The anti-SPINK1 mAb attenuated 22RV1 cell motility compared to IgG control, but had no effect on PC3 (SPINK1−/ETS−) cell motility (FIG. 8A).

Oncogenic Effects of SPINK1 in Part Through Interaction with EGFR

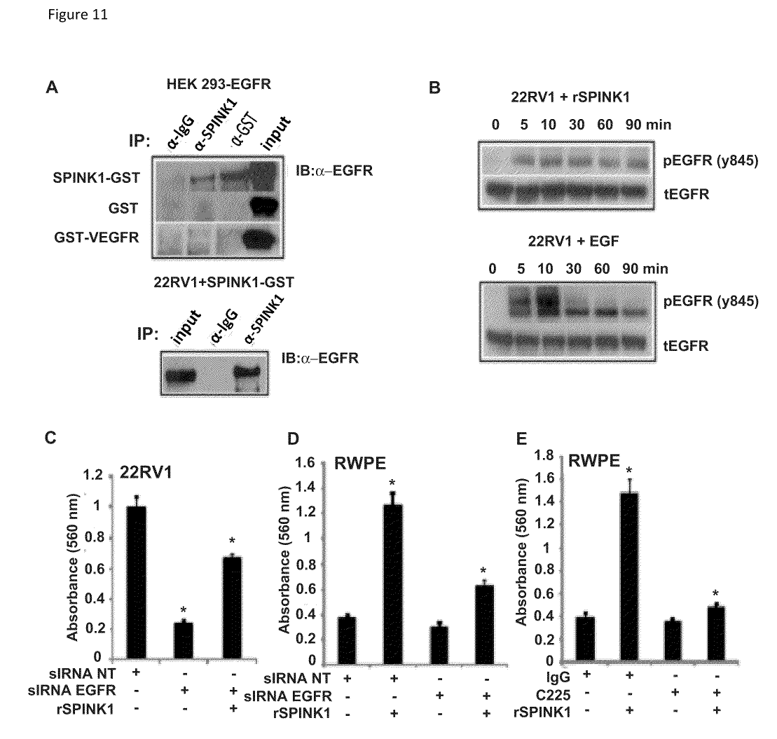

SPINK1 has a similar structure as epidermal growth factor (EGF), with approximately 50% sequence homology and three intrachain disulfide bridges (Hunt et al., Biochem Biophys Res Commun 60, 1020-1028 (1974); Bartelt et al., Arch Biochem Biophys 179, 189-199 (1977)). To characterize potential SPINK1 and EGFR interaction, EGFR was overexpressed EGFR in human embryonic kidney cells (HEK) 293 cells. The lysates were incubated with SPINK1-GST, GST or GST-VEGF Receptor 2 (GST-VEGFR) recombinant proteins. A strong interaction between SPINK1-GST and EGFR was observed but not with GST alone or GST-VEGFR recombinant protein (FIG. 11A; top panel).

Endogenous SPINK1 and EGFR interaction was not detected by immunoprecipitation and immunoblotting in 22RV1 cells, due to secretory nature of the SPINK1 protein. Addition of GST-SPINK1 to 22RV1 cells followed by immunoprecipitation and immunoblotting confirmed the interaction of SPINK1 and endogenous EGFR in 22RV1 cells (FIG. 11A; bottom panel).

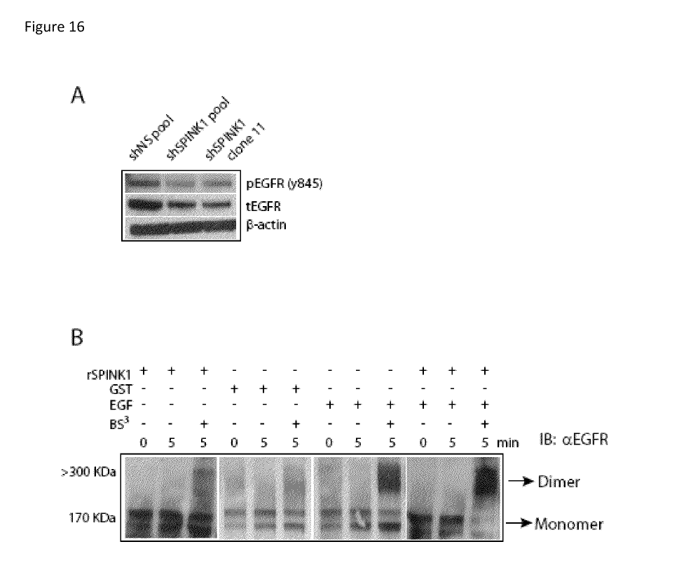

To further delineate the role of EGFR mediation of SPINK1 in prostate cancer, it was next assessed whether exogenous SPINK1 was capable of inducing EGFR phosphorylation (similar to the cognate ligand EGF). Stimulating 22RV1 cells with rSPINK1 resulted in EGFR phosphorylation, although weaker than that observed with EGF (FIG. 11B). rSPINK1 stimulation resulted in sustained EGFR phosphorylation over a 90 minute time course, while EGF resulted in strong EGFR phosphorylation which diminished after only 10 min. Similarly, stable shSPINKJ knockdown 22RV1 cells (pooled and clonal) showed decreased phosphorylated EGFR (pEGFR), with slightly decreased total EGFR (FIG. 16A). It was also demonstrated that rSPINK1 is able to induce dimerization of EGFR, although more weakly than EGF (FIG. 16B).

The functional consequences of SPINK1-EGFR interaction in the context of SPINK1+ prostate cancer was examined using 22RV1 cells. Transient knockdown of EGFR (FIG. 8B) blocked 22RV1 cell invasion by 75% (FIG. 11C; P=0.004) which was partially rescued by addition of exogenous SPINK1. A similar effect of EGFR knockdown was observed in RWPE cells treated with recombinant SPINK1 (FIG. 11D; P=0.014 and P=0.021 respectively). These results indicate that some of SPINK1's effects are mediated by EGFR.

It was next determined whether EGFR blockade could inhibit the oncogenic effects of SPINK1. It was first demonstrated that mAb to EGFR (cetuximab, C225) blocked the cell invasive effects of rSPINK1 in RWPE cells (FIG. 11E). C225 also blocked cell invasion of SPINK1+ 22RV1 cells but not in SPINK1-cell lines DU145, PC3, LNCaP or VCaP (FIG. 11F). Combining mAbs to SPINK1 and EGFR had an additive effect in the inhibition of 22RV1 cell invasion (FIG. 11G; P=0.001). In contrast to mAb to SPINK1 (FIG. 11A), C225 had no effect on 22RV1 cell proliferation or PC3 and DU145 cells proliferation (FIG. 11H). Together, these experiments indicate that SPINK1 has both EGFR-dependent and EGFR-independent functions in prostate cancer.

To investigate the downstream signaling pathways involved in the SPINK1-EGFR axis, the the mitogen-activated protein kinase (MAPK) and protein kinase B/AKT pathways were investigated in stable SPINK1 knockdown 22RV1 cells (shSPINK1 clone 11). Decreased pMEK, pERK and pAKT in stable shSPINK1 cells compared to control shNS cells was observed (FIG. 8C). Likewise, 22RV1 cells treated with SPINK1 mAb antibody showed decreased pERK (FIG. 8D).

### In Vivo Targeting of SPINK1

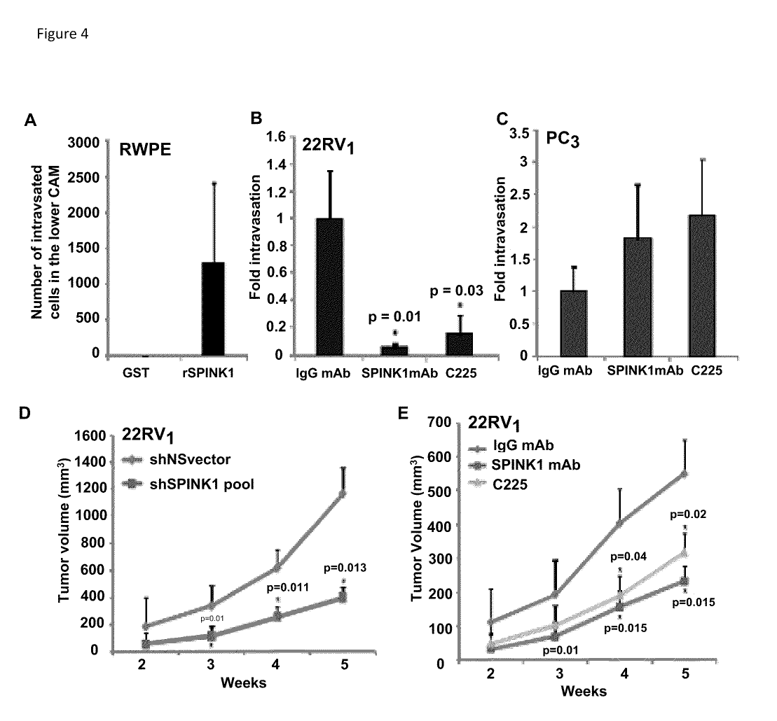

The in vitro studies showed that SPINK1 mediates cell proliferation and invasion in SPINK1⁺ prostate cancer cells. An in vivo model was used to assay a mAb for targeting SPINK1⁺ cancer cells in vivo. To qualify SPINK1 as a therapeutic target, shSPINK1-luciferase and shNS-luciferase vector cells were implanted in nude mice. At both 4 and 5 weeks 22RV1-shSPINK1-luciferase cells formed significantly smaller tumors (55% reduction at week 4; P=0.013 and 63% reduction at week 5; P=0.008) compared to shNS-luc vector cells (FIG. 4A). This effect is especially dramatic considering that a pooled population of shSPINK1 cells (and not a clone) was used.

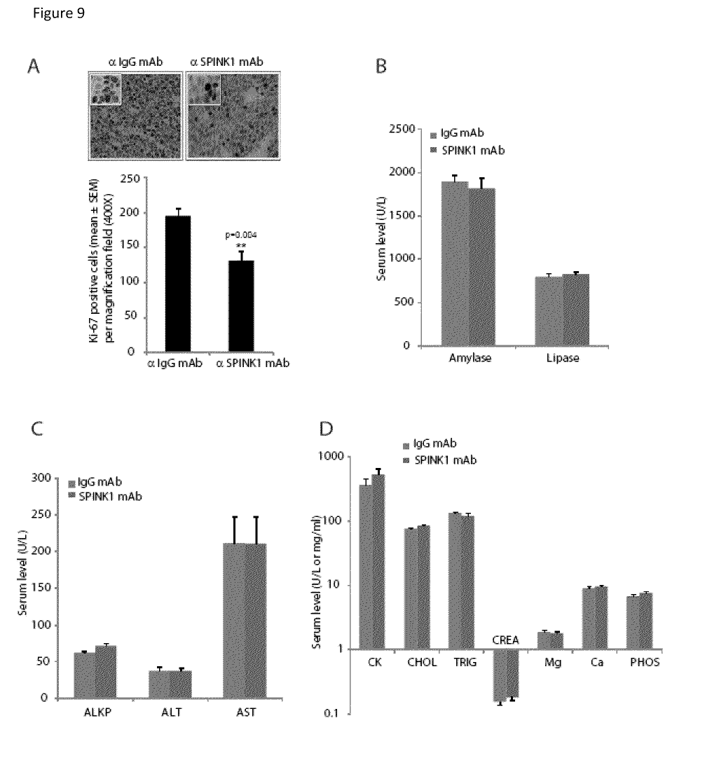

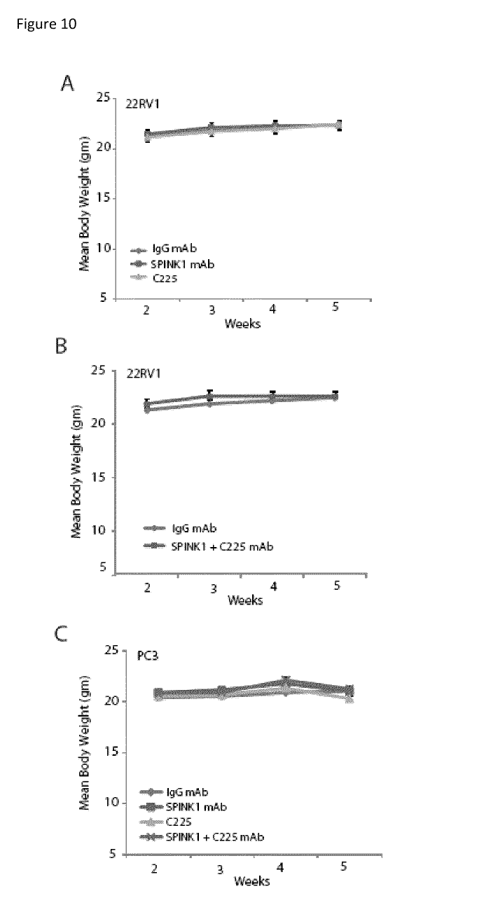

To demonstrate the preclinical efficacy of the anti-SPINK1 mAb, nude mice implanted with 22RV1-luciferase cells were treated with SPINK1 mAb (10 mg/kg body weight) or mouse monoclonal IgG (10 mg/kg body weight) twice a week. As shown in FIG. 4B, administration of SPINK1 mAb resulted in a 59% reduction of tumor burden at week 4 (P=0.015) and 55% reduction at week 5 (P=0.015). Similarly, there was a 60% reduction in tumor burden at week 4 as assessed by bioluminescence imaging. A significant decrease in Ki-67 positive nuclei and mitoses were recorded in the SPINK1 mAb treated group as compared to the control group (FIG. 9). There was no evidence of morbidity in either group, weekly body weights were similar in both groups, and there was no difference in mean amylase or lipase between treated and control groups (FIG. 10).

As SPINK1 mediates its oncogenic effects in part through EGFR, the mAb to EGFR (C225) was assessed using the same dosage schedule. C225 treatment resulted in a 41% reduction at week 4 (P=0.04) and 37% reduction at week 5 (P=0.02) (FIGS. 4E & I). By combining mAbs to SPINK1 and EGFR an additive effect was observed in vivo showing a 74% and 73% reduction in the growth of 22RV1 xenografts at week 4 (P=0.01) and 5 (P=0.003) respectively (FIGS. 4F & I). To confirm the in vitro results, which indicate no effect of SPINK1 or EGFR inhibition on SPINK1-prostate cancer, a xenograft study was performed using PC3 cells. Neither SPINK1 mAb nor C225 significantly inhibited tumor growth in PC3 xenografted mice (FIGS. 4G & 4I).

To investigate the role of SPINK1 in intravasation, a chick chorioallantoic membrane (CAM) model system was used (Zijlstra et al., Cancer Res 62, 7083-7092 (2002)) to demonstrate that rSPINK1 induced intravasation of benign RWPE cells (FIG. 4A). SPINK1 mAb and C225 significantly inhibit 22RV1 cell intravasation (P=0.01 and P=0.03 respectively), but did not significantly inhibit PC3 cell intravasation (FIGS. 4B & C).

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

We claim:

1. A method of inhibiting at least one biological function of a serine peptidase inhibitor, Kazal type I (SPINK1) polypeptide, comprising contacting said SPINK1 polypeptide with an antibody that specifically binds to said SPINK1 polypeptide and inhibits at least one biological function of said SPINK1 polypeptide.

2. The method of claim 1, wherein said SPINK1 is secreted by a cell.

3. The method of claim 2, wherein said cell is a cancer cell.

4. The method of claim 3, wherein said cancer cell is a prostate cancer cell.

5. The method of claim 2, wherein said cell is in vivo.

6. The method of claim 5, wherein said cell is in an animal.

7. The method of claim 6, wherein said animal is a human.

8. The method of claim 2, wherein said cell is ex vivo.

9. The method of claim 2, wherein said inhibiting at least one biological function of SPINK1 inhibits the proliferation of said cell.

10. The method of claim 2, wherein said inhibiting at least one biological function of SPINK1 inhibits the invasiveness of said cell.

11. The method of claim 2, further comprising the step of administering a second agent to said cell.

12. The method of claim 11, wherein said second agent inhibits at least one biological function of EGFR.

13. The method of claim 11, wherein said second agent is an anti-cancer therapeutic agent.

14. The method of claim 2, wherein said cell does not harbor an ETS gene fusion.

15. The method of claim 14, wherein said ETS gene fusion is a TMPRSS2:ETS gene fusion.

16. A kit, comprising a pharmaceutical composition that inhibits at least one biological function of SPINK1, wherein said composition comprises an antibody that specifically binds to SPINK1 and inhibits at least one biological function of SPINK1.

17. The kit of claim 16, further comprising a reagent that inhibits at least one biological function of EGFR.


SEQUENCE LISTING (31 sequences)

SEQ ID NO: 1 - DNA, 20 bp, Artificial Sequence
  Synthetic
tgtctgtgggactgatggaa

SEQ ID NO: 2 - DNA, 20 bp, Artificial Sequence
  Synthetic
aggcccagatttttgaatga

SEQ ID NO: 3 - DNA, 20 bp, Artificial Sequence
  Synthetic
tgcaccaccaactgcttagc

SEQ ID NO: 4 - DNA, 21 bp, Artificial Sequence
  Synthetic
ggcatggactgtggtcatgag

SEQ ID NO: 5 - RNA, 19 nt, Artificial Sequence
  Synthetic
ggaaauacuuaucccaaug

SEQ ID NO: 6 - RNA, 19 nt, Artificial Sequence
  Synthetic
uaauggaugcaccaagaua

SEQ ID NO: 7 - RNA, 19 nt, Artificial Sequence
  Synthetic
gaagagaggccaaauguua

SEQ ID NO: 8 - DNA, 20 bp, Artificial Sequence
  Synthetic
gcctggacgctcctgtgctg

SEQ ID NO: 9 - DNA, 20 bp, Artificial Sequence
  Synthetic
ctgggcacagccatcacccc

SEQ ID NO: 10 - DNA, 21 bp, Artificial Sequence
  Synthetic
gggccaggtcttgaaggctgt

SEQ ID NO: 11 - DNA, 20 bp, Artificial Sequence
  Synthetic
atccccagggccaccaccag

SEQ ID NO: 12 - RNA, 19 nt, Artificial Sequence
  Synthetic
cagaggauguucaauaacu

SEQ ID NO: 13 - protein, 56 aa, Artificial Sequence
  Synthetic
DSLGREAKCYNELNGCTKIYDPVCGTDGNTYPNECVLCFENRKRQTSILIQKSGPC

SEQ ID NO: 14 - protein, 14 aa, Artificial Sequence
  Synthetic
DSLGREAKCYNELN

SEQ ID NO: 15 - protein, 14 aa, Artificial Sequence
  Synthetic
KCYNELNGCTKIYD

SEQ ID NO: 16 - protein, 14 aa, Artificial Sequence
  Synthetic
GCTKIYDPVCGTDG

SEQ ID NO: 17 - protein, 14 aa, Artificial Sequence
  Synthetic
PVCGTDGNTYPNEC

SEQ ID NO: 18 - protein, 14 aa, Artificial Sequence
  Synthetic
NTYPNECVLCFENR

SEQ ID NO: 19 - protein, 14 aa, Artificial Sequence
  Synthetic
VLCFENRKRQTSIL

SEQ ID NO: 20 - protein, 14 aa, Artificial Sequence
  Synthetic
KRQTSILIQKSGPC

SEQ ID NO: 21 - protein, 42 aa, Artificial Sequence
  Synthetic
DSLGREAKCYNELNGCTKIYDPVCGTDGNTYPNECVLCFENR

SEQ ID NO: 22 - protein, 42 aa, Artificial Sequence
  Synthetic
KCYNELNGCTKIYDPVCGTDGNTYPNECVLCFENRKRQTSIL

SEQ ID NO: 23 - protein, 42 aa, Artificial Sequence
  Synthetic
GCTKIYDPVCGTDGNTYPNECVLCFENRKRQTSILIQKSGPC

SEQ ID NO: 24 - protein, 21 aa, Artificial Sequence
  Synthetic
DSLGREAKCYNELNGCTKIYD

SEQ ID NO: 25 - protein, 35 aa, Artificial Sequence
  Synthetic
PVCGTDGNTYPNECVLCFENRKRQTSILIQKSGPC

SEQ ID NO: 26 - protein, 28 aa, Artificial Sequence
  Synthetic
NTYPNECVLCFENRKRQTSILIQKSGPC

SEQ ID NO: 27 - protein, 21 aa, Artificial Sequence
  Synthetic
VLCFENRKRQTSILIQKSGPC

SEQ ID NO: 28 - protein, 28 aa, Artificial Sequence
  Synthetic
GCTKIYDPVCGTDGNTYPNECVLCFENR

SEQ ID NO: 29 - protein, 35 aa, Artificial Sequence
  Synthetic
GCTKIYDPVCGTDGNTYPNECVLCFENRKRQTSIL

SEQ ID NO: 30 - protein, 21 aa, Artificial Sequence
  Synthetic
GCTKIYDPVCGTDGNTYPNEC

SEQ ID NO: 31 - protein, 21 aa, Artificial Sequence
  Synthetic
PVCGTDGNTYPNECVLCFENR